SEQUENCE LISTING (6 sequences)

SEQ ID NO: 1 - protein, 25 aa, Unknown
  Description of Unknown RCL sequence
GTEAAGAMFLEAIPMSIPPEVKFNK

SEQ ID NO: 2 - protein, 16 aa, Artificial Sequence
  Description of Artificial Sequence Synthetic peptide
DTEEEDFHVDQVTTVK

SEQ ID NO: 3 - protein, 8 aa, Unknown
  Description of Unknown Adjacent RCL Tryptic sequence
AVLTIDEK

SEQ ID NO: 4 - protein, 15 aa, Unknown
  Description of Unknown RCL Cleaved sequence
GTEAAGAMFLEAIPM

SEQ ID NO: 5 - protein, 22 aa, Unknown
  Description of Unknown RCL Intact sequence
GTEAAGAMFLEAIPMSIPPEVK

SEQ ID NO: 6 - protein, 7 aa, Unknown
  Description of Unknown RCL Cleaved sequence
SIPPEVK

(19) United States
(12) Patent Application Publication
(10) Pub. No.: US 2018/0306798 A1
(43) Pub. Date: Oct. 25, 2018

(54) Monitoring Dysregulated Serum Complement, Coagulation, and Acute-Phase Inflammation Sub-Proteomes Associated with Cancer

(71) Applicant: Biotech Support Group LLC, Monmouth Junction, NJ (US)

(72) Inventors: Swapan Roy, East Brunswick, NJ (US); Devjit Roy, Casper, WY (US); Matthew Kuruc, Leonia, NJ (US)

(21) Appl. No.: 15/953,260

(22) Filed: Apr. 13, 2018

Related U.S. Application Data
(60) Provisional application No. 62/485,868, filed on Apr. 14, 2017.

(51) Int. Cl. G01N 33/574; G01N 33/50; G01N 33/566; G06F 19/12; G06F 19/18; G06F 19/28

(52) U.S. Cl. CPC G01N 33/57488; G01N 33/57423; G01N 33/5091; G01N 33/566; G01N 33/57438; G01N 33/57415; G01N 2800/60; G06F 19/18; G06F 19/28; G01N 2033/57461; G01N 2033/57403; G01N 2033/57453; G06F 19/12

(57) ABSTRACT

Embodiments include obtaining a stroma liquid biopsy from a patient and detecting a pattern of dysregulation amongst the biomarkers in the stroma liquid biopsy that can be monitored to help screen, diagnose or treat the patient for cancer. The biomarkers in the stroma liquid biopsy are involved in interconnected pathways such as the coagulation pathway, acute-phase inflammation pathway, and complement pathway. Particular embodiments involve assaying for levels of biomarkers, such as neutrophil elastase, as well as subpopulations of other biomarkers, such as subpopulations of SERPIN proteins. The levels of biomarkers and/or the ratios of levels of biomarker subpopulations can be informative for predicting the cancer disease state in the patient, thereby enabling more personalized or tailored medical intervention for the patient.

### CROSS REFERENCE TO RELATED APPLICATIONS

This application claims the benefit of U.S. Provisional Application No. 62/485,868, filed on Apr. 14, 2017, which is herein incorporated by reference in its entirety for all purposes.

### BACKGROUND

The proteome can include the entire set of proteins, produced or modified by a subject. Unlike the genome, the proteome varies with time and environmental stresses so that a subject's proteome is dynamic. Proteomics is an interdisciplinary field utilizing the basic genome information as a correlating attribute, while exploring and characterizing the vast information contained in protein composition, structure, and function. The advancements in genome sequencing has spawned efforts to utilize biopsy products and services that probe the proteome of a subject. These conventional techniques for characterizing cancers, examples of which include liquid or tumor biopsies, only focus on analyzing the tumor cell genome. Challenges remain as genomic instability is a fundamental hallmark of cancer cells. In many respects, genomic driven approaches suffer the problem of shooting after a moving target.

### SUMMARY

Described herein are methods that include obtaining a stroma liquid biopsy from a patient and detecting a pattern of dysregulation amongst the biomarkers in the stroma liquid biopsy that can be monitored to help screen, diagnose or treat the patient for cancer. The stroma liquid biopsy is informative for understanding the stroma microenvironment surrounding a cancer, which is a diverse milieu of soluble and membrane-bound proteins mediating multiple cellular components and biological processes. Reciprocal communication between tumor and healthy cells in the stroma microenvironment regulates the diverse components of the extracellular matrix, ultimately promoting tumor growth, survival and eventual colonization to metastatic sites. Certain tissue microenvironments may be especially hospitable to early disease or to new metastatic lesions. Consequently, metastatic potential is likely to depend upon some of the same supportive mechanisms in the microenvironments of the stroma for the hospitality needed for reseeding and colonization by circulating cancer cells from the primary tumor. As stromal cells within the microenvironments of tumors are genetically stable compared to tumor cells, their derivative proteomes may offer attractive therapeutic targets to help manage an often incurable disease and prolong survival. As such, any and all multi-parametric profiles that can help to monitor and/or stratify cancer patients for individual clinical situations will become desirable.

Here, several components of cancer dysregulation measurable from the tumor-associated stromal microenvironment commonly obtained from whole blood, either serum or plasma are identified. Embodiments of the invention disclose a pattern of biomarker levels detected in a patient's stroma liquid biopsy, which can be measured and modeled for the management and treatment of cancer patients, without regard to the primary tumor of origin, clinical stage of disease, or tumor burden. Embodiments of the invention are orthogonal to and complementary with, liquid biopsy technologies based on ‘nextgen’ sequencing, circulating tumor cells (CTCs), circulating DNA (ctDNA), circulating extracellular vesicles (exosomes), and tumor burden biomarkers (i.e. CEA, CA125). This new observational window can help generate a more comprehensive profile of progressive disease, providing opportunities in monitoring risk factors, early detection, prognosis, recurrence, and guidance for therapeutic decisions.

As disclosed herein, certain embodiments describe a method for treating a cancer subject, the method comprising: obtaining a dataset comprising levels of two or more biomarker proteins in a sample obtained from the cancer subject, the two or more biomarker proteins involved in two or more interconnected pathways of dysregulation or systemic regulation of the two or more interconnected pathways of dysregulation, the two or more interconnected pathways comprising a coagulation pathway, a complement pathway, and an acute-phase inflammation pathway; determining a disease state of the cancer based on the detected levels of the biomarker proteins; and based on the determined disease state of the cancer, administering a therapeutic compound that modulates one or more of the detected levels of the biomarker proteins towards corresponding levels of the biomarker proteins that are exhibited by healthy subjects, wherein biomarker proteins involved in the coagulation pathway comprise tissue inhibitor of metalloproteinases-1 (TIMP1), Pro-platelet basic protein (PPBP), thrombospondin 1 (THBS1), platelet Factor 4 (PF4), and an active subpopulation of heparin cofactor 2 (HEP2), wherein biomarker proteins involved in the complement pathway comprise complement (C3), complement component 4 binding protein alpha (C4BPA), properdin (PROP), wherein biomarker proteins involved in the acute-phase inflammation pathway comprise, Serum Amyloid 2 (SAA2), extracellular matrix protein 1 (ECM1), Neutrophil Elastase (ELANE), and chromogranin A (CMGA), wherein biomarker proteins involved in the systemic regulation of the coagulation, complement, and acute-phase inflammation pathways comprise one or more serine proteinase inhibitor (SERPIN) proteins.

In various embodiments, the one or more SERPIN proteins comprise alpha-1-antitrypsin (SERPINA1), wherein determining the disease state of the cancer based on the detected levels of the biomarker proteins comprises: determining a ratio of a detected level of an inactive subpopulation of SERPINA1 and a detected level of an active subpopulation of SERPINA1; and determining that the determined ratio is elevated in comparison to a corresponding ratio of a level of an inactive subpopulation of SERPINA1 and a level of an active subpopulation of SERPINA1 detected in samples obtained from healthy subjects, wherein the determined ratio is at least 3.5 times greater than the corresponding ratio detected in samples obtained from healthy subjects.

In various embodiments, the one or more SERPIN proteins are antichymotrypsin (SERPINA3), plasma protease C1 inhibitor (SERPING1), heparin cofactor II (SERPIND1), antithrombin III (SERPINC1), alpha-1-antitrypsin (SERPINA1), kallistatin (SERPINA4), protein C inhibitor (SERPINA5), Z-dependent proteinase inhibitor (SERPINA10), and alpha-2-antiplasmin (SERPINF2). In various embodiments, determining the disease state of the cancer based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects. In various embodiments, determining the disease state of the cancer based on the detected levels of the biomarker proteins comprises determining that a detected level of an inactive subpopulation of a SERPIN protein is at least 1.5 times greater or 1.5 times less than a level of an inactive subpopulation of a SERPIN protein detected in samples obtained from healthy subjects.

In various embodiments, determining the disease state of the cancer based on the detected levels of the biomarker proteins comprises determining a ratio of a detected level of ELANE to one of a detected level of an inactive subpopulation of SERPINA1 or a detected level of an active subpopulation of SERPINA1. In various embodiments, the determined ratio of the detected level of ELANE to the detected level of the active subpopulation of SERPINA1 is at least 10 times greater than the corresponding ratio detected in samples obtained from healthy subjects.

In various embodiments determining the disease state of the cancer based on the detected levels of the biomarker proteins further comprises determining that the detected levels of one or more of THBS1, TIMP1, PPBP, or PF4 are elevated, and the active subpopulation of HEP2 is lower in comparison to corresponding levels detected in samples obtained from healthy subjects. In various embodiments, determining the disease state of the cancer based on the detected levels of the biomarker proteins further comprises determining that the detected levels of one or more of THBS1, TIMP1, PPBP, or PF4 are at least 10 times greater, and the active subpopulation HEP2 is at least 1.5 times lower in comparison to corresponding levels detected in samples obtained from healthy subjects.

In various embodiments, determining the disease state of the cancer based on the detected levels of the biomarker proteins further comprises determining that the detected levels of one or more of C3, C4BPA, or PROP are lower in comparison to corresponding levels detected in samples obtained from healthy subjects. In some embodiments, determining the disease state of the cancer based on the detected levels of the biomarker proteins further comprises determining that the detected levels of one or more of C3, C4BPA, or PROP are at least 1.5 times lower in comparison to corresponding levels detected in samples obtained from healthy subjects.

In various embodiments, determining the disease state of the cancer based on the detected levels of the biomarker proteins comprises determining that a detected level of one or more of SAA2, ECM1, ELANE, and CMGA are elevated in comparison to corresponding levels detected in samples obtained from healthy subjects. In some embodiments, determining the disease state of the cancer based on the detected levels of the biomarker proteins comprises determining that a detected level of one or more of SAA2 and ECM1 are elevated at least 1.5 times, ELANE is elevated at least 2 times, and CMGA is elevated at least 10 times in comparison to corresponding levels detected in samples obtained from healthy subjects.

As further disclosed herein, certain embodiments describe a method for determining or diagnosing presence of cancer or risk factors for cancer in a subject, the method comprising: obtaining a dataset comprising levels of two or more biomarker proteins in a sample obtained from the cancer subject, the two or more biomarker proteins involved in two or more interconnected pathways of dysregulation or systemic regulation of the two or more interconnected pathways of dysregulation, the two or more interconnected pathways comprising a coagulation pathway, a complement pathway, and an acute-phase inflammation pathway; determining or diagnosing presence of cancer or risk factors for cancer in the subject based on the detected levels of the biomarker proteins, wherein biomarker proteins involved in the coagulation pathway comprise tissue inhibitor of metalloproteinases-1 (TIMP1), Pro-platelet basic protein (PPBP), thrombospondin 1 (THBS1), platelet Factor 4 (PF4), and an active subpopulation of heparin cofactor 2 (HEP2), wherein biomarker proteins involved in the complement pathway comprise complement (C3), complement component 4 binding protein alpha (C4BPA), properdin (PROP), wherein biomarker proteins involved in the acute-phase inflammation pathway comprise, Serum Amyloid 2 (SAA2), extracellular matrix protein 1 (ECM1), Neutrophil Elastase (ELANE), and chromogranin A (CMGA), wherein biomarker proteins involved in the systemic regulation of the coagulation, complement, and acute-phase inflammation pathways comprise one or more serine proteinase inhibitor (SERPIN) proteins.

In various embodiments, the one or more SERPIN proteins comprise alpha-1-antitrypsin (SERPINA1), wherein determining or diagnosing presence of cancer or risk factors for in the subject based on the detected levels of the biomarker proteins comprises: determining a ratio of a detected level of an inactive subpopulation of SERPINA1 and a detected level of an active subpopulation of SERPINA1; and determining that the determined ratio is elevated in comparison to a corresponding ratio of a level of an inactive subpopulation of SERPINA1 and a level of an active subpopulation of SERPINA1 detected in samples obtained from healthy subjects, wherein the determined ratio is at least 3.5 times greater than the corresponding ratio detected in samples obtained from healthy subjects.

In various embodiments, the one or more SERPIN proteins are antichymotrypsin (SERPINA3), plasma protease C1 inhibitor (SERPING1), heparin cofactor II (SERPIND1), antithrombin III (SERPINC1), alpha-1-antitrypsin (SERPINA1), kallistatin (SERPINA4), protein C inhibitor (SERPINA5), Z-dependent proteinase inhibitor (SERPINA10), and alpha-2-antiplasmin (SERPINF2).

In various embodiments, determining or diagnosing presence of cancer or risk factors for cancer in the subject based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects. In some embodiments, determining or diagnosing presence of cancer or risk factors for cancer in the subject based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects.

In various embodiments, determining or diagnosing presence of cancer or risk factors for cancer in the subject based on the detected levels of the biomarker proteins comprises determining a ratio of a detected level of ELANE to one of a detected level of an inactive subpopulation of SERPINA1 or a detected level of an active subpopulation of SERPINA1. In some embodiments, wherein the determined ratio of the detected level of ELANE to the detected level of the active subpopulation of SERPINA1 is at least 10 times greater than the corresponding ratio detected in samples obtained from healthy subjects.

In various embodiments, determining or diagnosing presence of cancer or risk factors for cancer in the subject based on the detected levels of the biomarker proteins comprises determining that the detected levels of one or more of THBS1, TIMP1, PPBP, or PF4 are elevated, and the active subpopulation of HEP2 is lower in comparison to corresponding levels detected in samples obtained from healthy subjects. In some embodiments, determining or diagnosing presence of cancer or risk factors for cancer in the subject based on the detected levels of the biomarker proteins comprises determining that the detected levels of one or more of THBS1, TIMP1, PPBP, or PF4 are at least 10 times greater, and the active subpopulation of HEP2 is at least 1.5 times lower in comparison to corresponding levels detected in samples obtained from healthy subjects.

In various embodiments, determining or diagnosing presence of cancer or risk factors for cancer in the subject based on the detected levels of the biomarker proteins comprises determining that the detected levels of one or more of C3, C4BPA, or PROP are lower in comparison to corresponding levels detected in samples obtained from healthy subjects. In some embodiments, determining or diagnosing presence of cancer or risk factors for cancer in the subject based on the detected levels of the biomarker proteins comprises determining that the detected levels of one or more of C3, C4BPA, or PROP are at least 1.5 times lower in comparison to corresponding levels detected in samples obtained from healthy subjects.

In various embodiments, determining or diagnosing presence of cancer or risk factors for cancer in the subject based on the detected levels of the biomarker proteins comprises determining that a detected level of one or more of SAA2, ECM1, ELANE and CMGA are elevated in comparison to corresponding levels detected in samples obtained from healthy subjects. In some embodiments, determining or diagnosing presence of cancer or risk factors for cancer in the subject based on the detected levels of the biomarker proteins comprises determining that a detected level of one or more of SAA2 and ECM1 are elevated at least 1.5 times, ELANE is elevated at least 2 times, and CMGA is elevated at least 10 times in comparison to corresponding levels detected in samples obtained from healthy subjects.

### BRIEF DESCRIPTION OF THE DRAWINGS

The accompanying drawing(s), which are incorporated in and constitute a part of this specification, illustrate several aspects described below.

FIG. 1A depicts an overall environment for characterizing a cancer state in a cancer patient, in accordance with an embodiment.

FIG. 1B depicts a flow process of characterizing a cancer state in a cancer patient and treating the cancer patient based on the characterized cancer state, in accordance with an embodiment.

FIG. 1C depicts an example computer, in accordance with an embodiment.

FIG. 2 depicts the interplay between SERPIN proteins in three interconnected pathways.

FIG. 3 depicts quantified levels of bead bound active AAT and total AAT load in lung, breast, and pancreatic cancers.

FIG. 4 depicts signal intensity representing total AAT load detected through a protein surrogate in patients with breast, lung, or pancreatic cancer.

FIG. 5 depicts signal intensity representing total AAT load and flow-through inactive AAT in patients with breast, lung, or pancreatic cancer.

FIG. 6 depicts normalized levels of total AAT load and inactive AAT in patients with breast, lung, or pancreatic cancer in relation to healthy patients.

FIG. 7 depicts normalized levels of total AAT load and bead-bound active AAT in patients with breast, lung, or pancreatic cancer in relation to healthy patients.

FIG. 8 depicts a normalized total AAT load and normalized ratios of inactive AAT to active AAT in patients with breast, lung, or pancreatic cancer in relation to healthy patients.

FIG. 9 depicts signal intensity representing total Neutrophil Elastase in patients with breast, lung, or pancreatic cancer or healthy patients.

FIG. 10 depicts normalized ratios of Neutrophil Elastase to bead-bound active AAT in patients with breast, lung, or pancreatic cancer in relation to healthy patients.

### DETAILED DESCRIPTION

### 1.1. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The detailed description of the invention is divided into various sections only for the reader's convenience, and disclosure found in any section may be combined with that in another section. Unless defined otherwise, all technical and scientific terms used herein have the same meaning as commonly understood by one of ordinary skill in the art to which this invention belongs.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

In this disclosure, “comprises,” “comprising,” “containing,” “having,” “includes,” “including,” and linguistic variants thereof have the meaning ascribed to them in U.S. patent law, permitting the presence of additional components beyond those explicitly recited.

Unless specifically stated or otherwise apparent from context, as used herein the term “about” or “approximately” is understood as within a range of normal tolerance in the art, for example within 2 standard deviations of the mean and is meant to encompass variations of ±20% or ±10%, more preferably ±5%, even more preferably ±1%, and still more preferably ±0.1% from the stated value.

The terms “marker,” “markers,” “biomarker,” and “biomarkers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.).

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female.

The term “obtaining a dataset” encompasses obtaining a set of data determined from at least one sample. Obtaining a dataset encompasses obtaining a sample, and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

The term “proteoform” encompasses a conformation variant of the full length polypeptide sequence because of post-translational modification. In this disclosure, proteoform describes protein variants as they relate to the hydrolysis of amino acids at specific amino acid sequences, or cleavage sites of the full length protein.

The term “RCL” or “Reactive Centre Loop” within the SERPIN family of protease inhibitors, describes a specific amino acid region about 6-20 amino acids in length that covalently interacts with a protease substrate, or is nevertheless hydrolyzed or cleaved at specific sites within the region.

In this disclosure the Uniprot.org database annotations of biomarker proteins are adopted in parentheses.

### 1.2. Methods for Determining a Cancer Disease State in a Subject

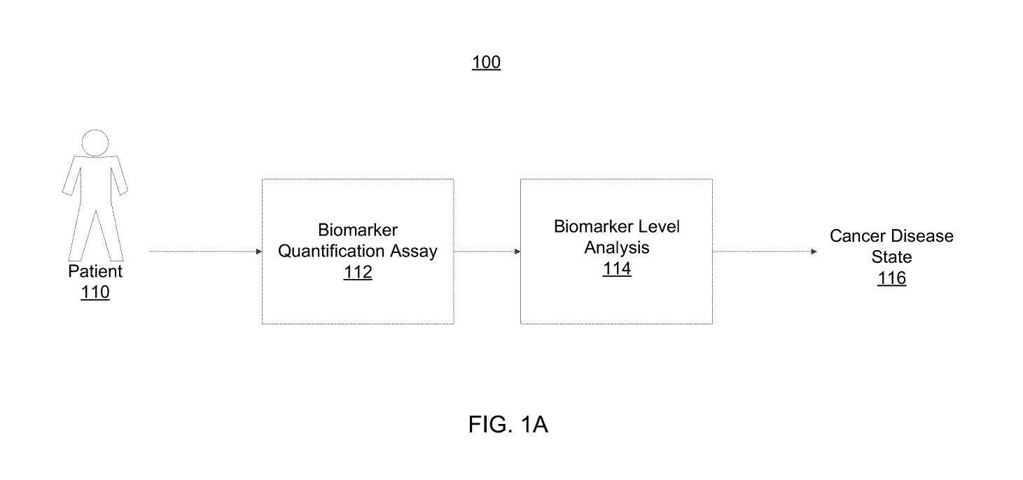

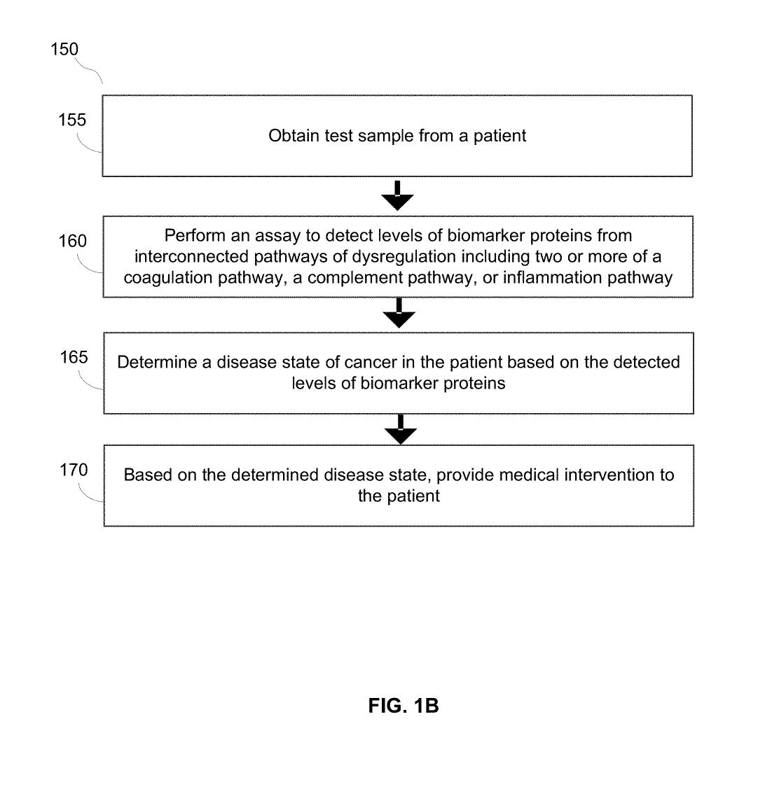

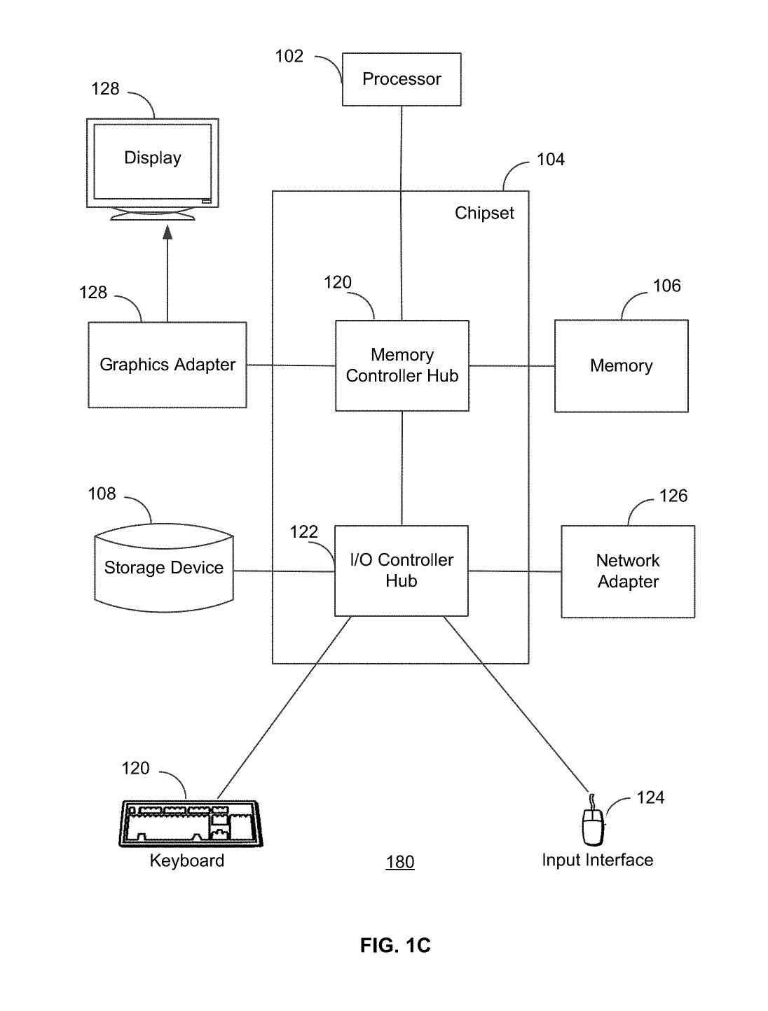

Disclosed herein are methods for determining a cancer disease state in a subject. In accordance with one embodiment, FIG. 1A depicts an overview environment 100 for determining a cancer disease state in a subject. The environment 100 provides context in order to introduce a patient 110, biomarker quantification assay 112, a biomarker analysis 114, and a cancer disease state 116. Additional reference will be made to FIG. 1B, which depicts an example flow process 150 for determining a cancer disease state in a subject.

At step 155 shown in FIG. 1B, a test sample is obtained from a patient 110. The test sample can be obtained by the patient 110 or by a third party, e.g., a medical professional. Examples of medical professionals include physicians, emergency medical technicians, nurses, first responders, psychologists, medical physics personnel, nurse practitioners, surgeons, dentists, and any other obvious medical professional as would be known to one skilled in the art. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the test sample is obtained by a blood draw, where the medical professional draws blood from a subject, such as by a syringe.

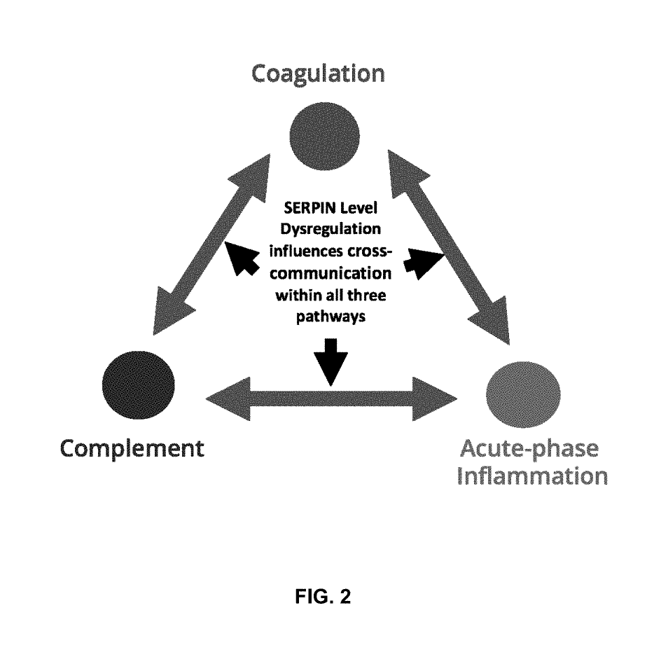

Step 160 includes the performance of an assay, hereafter generally referred to as a biomarker quantification assay 112, to detect levels of biomarker proteins that are involved in interconnected pathways of dysregulation, examples of which include the coagulation pathway, acute-inflammation pathway, complement pathway, or glycolysis pathway. The biomarker quantification assay 112 determines quantitative values of one or more biomarkers from a test sample obtained from the patient 110. In particular embodiments, the biomarker quantification assay 112 is a liquid chromatography-mass spectrometry (LC-MS) assay or a liquid chromatography-tandem mass spectrometry (LC-MS/MS). In other embodiments, as discussed in further detail below, other types of assays are applied to determine quantitative values of one or more biomarkers from the test sample. Biomarkers involved in one or more dysregulated pathways of cancer are described in further detail below in relation to FIG. 2.

At step 165, a disease state of the cancer in the patient is determined based on the detected levels of biomarker proteins. For example, referring to FIG. 1A, the biomarker level analysis 114 evaluates the quantitative biomarker levels of the biomarkers determined by the biomarker quantification assay 112 and outputs a cancer disease state 116 (e.g., interchangeably referred to as a “disease state of the cancer”). In various embodiments, the biomarker level analysis 114 includes a prediction model that is executed by one or more processors of a computer X, an example of which is shown below with respect to FIG. X. In these embodiments, the prediction model outputs the cancer disease state 116.

In various embodiments, the cancer disease state 116 refers to a disease state of the cancer in the patient 110. Such a disease state can be in relation to or independent of a primary tumor of origin, a clinical stage of disease, or tumor burden. In various embodiments, the disease state of the cancer is one of a presence or absence of cancer in the patient 110. In various embodiments, the disease state of the cancer is a severity (e.g., a grade) of a cancer in the patient 110. In various embodiments, the disease state of the cancer refers to the likelihood that the patient 110 develops cancer in the future. In various embodiments, the disease state of the cancer refers to the presence of risk factors in the patient 110 that contribute towards a likelihood of developing cancer. In various embodiments, the disease state of the cancer refers to a stratification of the patient based on the quantitative biomarker levels detected in the test sample obtained from the patient. In various embodiments, the cancer disease state 116 output by the prediction model is a total score that represents a disease state of the cancer in the patient 110. For example, the prediction model may output a total score that represents one of how likely the cancer is present in the patient 110, a likelihood that the patient 110 develops cancer, or a likelihood of the presence of risk factors in the patient 110. As another example, the prediction model may output a total score that represents whether the patient 110 should be categorized in a particular stratification.

At step 170, based on the determined cancer disease state 116, the patient 110 can be provided medical intervention. For example, based on a detected presence of cancer in the patient 110, the patient 110 can be administered a therapeutic that treats the cancer in the patient 110. As another example, based on a stratification of the patient, the patient 110 can be administered a therapeutic that is deemed appropriate for patients categorized in the stratification. In one embodiment, the patient 110 can be administered a therapeutic that modulates levels of biomarkers that are expressed by the patient. For example, the therapeutic can modulate the levels of biomarkers that are involved in one or more dysregulated pathways of cancer, examples of which include the coagulation, acute-inflammation, complement, or glycolysis pathways.

1.2.1. Biomarker Quantification Assay

In particular embodiments, an assay used for detecting quantitative levels of one or more biomarkers involved in dysregulated pathways related to cancer is a Liquid Chromatography coupled to Mass Spectrometry (LC-MS or LC-MS/MS) assay. In various embodiments, examples of assays for detecting quantitative levels of one or more biomarkers include two dimensional polyacrylamide gel electrophoresis (2DPAGE & 2D-DIGE), surface or matrix enhanced laser desorption/ionization time of flight (SELDI-ToF, MALDI-ToF), protein antibody-capture arrays, multidimensional protein identification technology (MudPIT), microarrays, high performance liquid chromatography (HPLC), enzymatic assays, functional activity assays, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays. The information can also be qualitative, such as observing patterns or fluorescence, which can be translated into a quantitative measure by a user or automatically by a reader or computer system.

Various immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

In various embodiments, protein based analysis, which employs an antibody that specifically binds to a polypeptide (e.g. marker), can be used to quantify the marker level in a test sample obtained from an individual. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five or more markers.

In particular embodiments, the biomarker quantification assay further includes a protein level separation technique. Such a protein level separation technique can be performed prior to performing an assay for detecting quantitative levels of one or more biomarkers. In one embodiment, a protein level separation technique can include the employment of an albumin depletion kit, an example of which is the ALBUVOID LC-MS On-Bead for Serum Proteomics. The protein level separation technique can enrich low concentration biomarkers by depleting masking proteins such as albumin. Thus, in various embodiments, protein level separation of a sample can be performed using the ALBUVOID on-bead reagent kit to obtain a bead bound subpopulation and a flow-through (e.g., not bound to a bead) subpopulation. Here, the bead-bound subpopulation can represent proteins, an example of which is an active subpopulation of AAT, that exhibit a binding bias towards the ALBUVOID bead. The bead bound subpopulation can be later obtained by performing trypsin digestion of the bead bound proteins. Additionally, the flow-through subpopulation can represent proteins, an example of which is an inactive subpopulation of AAT, that do not exhibit a binding bias towards the ALBUVOID bead. The trypsinized bead bound subpopulation and the flow-through subpopulation can be separately quantified using the aforementioned assays (e.g., LC-MS or LC-MS/MS) to determine concentrations of particular biomarkers in each subpopulation.

1.2.2. Biomarkers

In some embodiments, the quantitative levels of one or more biomarkers are detected from a sample obtained from an individual. The values of one or more markers can be indicated as a numerical value. The numerical values can be obtained, for example, by experimentally obtaining measures from a sample obtained from an individual by an assay (e.g., an LC-MS assay) performed in a laboratory. Alternatively, numerical values of biomarkers can be included in a dataset obtained from a service provider such as a laboratory, or from a database or a server on which the dataset has been stored, e.g., on a storage memory. In an embodiment, numerical values of two, three, four, or more biomarkers can be included in the dataset associated with a test sample obtained from a subject. Such quantitative biomarker levels can then be used to predict a cancer disease state 116.

In some embodiments, the quantitative levels of one or more markers can be quantitative expression values of a first subpopulation of a SERPIN protein and a second subpopulation of a SERPIN protein. This is contrary to prior efforts that focus on detecting the AAT population as a whole (as opposed to subpopulations). Examples of a first subpopulation and a second population can be an active conformation and an inactive conformation of a SERPIN protein. In particular embodiments, the quantitative levels of one or more markers can be: a total population of Alpha-1-antitrypsin (AAT), an active subpopulation of AAT, an inactive subpopulation of AAT, and neutrophil elastase (ELANE). As used hereafter, an active subpopulation of AAT refers to AAT with an intact reactive centre loop (RCL) whereas an inactive subpopulation of AAT refers to AAT with a cleaved RCL.

In an embodiment, the quantity of one or more markers can be one or more quantitative expression values of: Tissue Inhibitor of Metalloproteinases 1 (TIMP1), Pro-platelet Basic Protein (PPBP), Platelet Factor 4 (PF4), Thrombospondin 1 (THBS1), Heparin Cofactor II (SERPIND1, also referred to as HEP2), Extracellular Matrix Protein 1 (ECM1), Complement Component 3 (C3), C4b-binding protein alpha chain (C4BPA), Complement Factor Properdin (CFP), Serum Amyloid A2 (SAA2), Chromogranin-A (CHGA), Fibronectin (FN1), Pregnancy Zone Protein (PZP), Antichymotrypsin (SERPINA3), Plasma Protease C1 Inhibitor (SERPING1), Antithrombin ATIII (SERPINC1), Kallistatin (SERPINA4), Protein C Inhibitor (SERPINA5), Z-dependent proteinase inhibitor (SERPINA10), α-2-antiplasmin (SERPINF2), inter alpha trypsin inhibitor heavy chain H1 (ITIH1), ITIH heavy chain H2 (ITIH2), ITIH heavy chain H3 (ITIH3), ITIH heavy chain H4 (ITIH4), Apolipoprotein A1 (APOA1), Apolipoprotein C III (APOC3), C-reactive protein (CRP), Clusterin (CLU), Polymeric immunoglobulin receptor (PIGR), Neutrophil-activating peptide 2 (NAP-2), complement component 1q (C1q), complement 1 (C1), complement C2 (C2), complement C4(a) subunit (C4a), complement C5 (C5), Transthyretin (TTR), Angiotensinogen (AGT), Carboxypeptidase N (CPN1), Immunoglobulin lambda variable 3-9 (IGLV3-9), immunoglobulin Heavy Variable 1/OR15-1 (IGHV1OR15-1), Immunoglobulin Heavy Variable 3-53 (IGHV3-53), Immunoglobulin Kappa Variable 1D-33 (IGKV1D-33), Sex hormone binding globulin (SHBG), Semaphorin 3D (SEMA3D), Cilia- and flagella-associated protein 61 (CFAP61), Phosphofructokinase 1 (PFKM), Sprouty related EVH1 domain containing 2, (SPRED2), Chromosome 18 Open Reading Frame 63 (C18orf63), Immunoglobulin Lambda Variable 3-27 (IGLV3-27), kininogen 1 (KNG1), kallikreins (KLKB1), and prostate specific antigen (PSA). Markers can also include those listed in the Tables and Figures.

In various embodiments, the quantity of one or more biomarkers can be one or more quantitative expression values of biomarkers that are involved in multiple dysregulated pathways of cancer. As an example, FIG. 2 depicts the interplay between SERPIN proteins in three interconnected pathways including the coagulation pathway, inflammation pathway, complement pathway. Specifically, SERPIN proteins are involved in the systemic regulation of each of the coagulation pathway, inflammation pathway, and complement pathway. In other words, the SERPIN proteins play a role in modulating and/or maintaining levels of different biomarkers that are involved in the coagulation pathway, inflammation pathway, and complement pathways. The SERPIN proteins include SERPINA3, SERPING1, SERPIND1, SERPINC1, SERPINA4, SERPINA5, SERPINA10, SERPINF2, SERPINA1, as well as their various subforms (e.g., active SERPINA1 and/or inactive SERPINA1). In various embodiments, additional interconnected, dysregulated pathways than shown in FIG. 2 may be involved in cancer and therefore, can be monitored through additional biomarkers than shown in FIG. 2.

Referring first to the coagulation pathway, it is the process by which blood changes from a liquid to a gel, forming a clot. That the coagulation system conspires in support of cancer progression serves to illustrate a normal homeostatic function being dysregulated in cancer pathogenesis. In various embodiments, the biomarker proteins involved in the pattern of dysregulation in the coagulation pathway include, but are not limited to, tissue inhibitor of metalloproteinases-1 (TIMP1), Pro-platelet basic protein (PPBP), thrombospondin 1 (THBS1), platelet Factor 4 (PF4), and an active subpopulation of heparin cofactor 2 (HEP2), Kininogen 1 (KNG1), Angiotensinogen (AGT), and C1 inhibitor (SERPING1). Kininogen has two splice variant isoforms, low molecular weight (LMWK) and high molecular weight (HMWK) derived from the same KNG1 gene. LMWK is not involved with coagulation whereas HMWK kininogen is implicated in the coagulation pathway.

While the activation of platelets is a downstream event, the coagulation cascade has two initial pathways which ultimately leads to the final clot formation. These are the contact system pathway (also known as the intrinsic pathway), and the tissue factor pathway (also known as the extrinsic pathway) which both activate the final common pathway of factor X, thrombin and fibrin. Plasma kallikrein is a protease involved in the contact system coagulation cascade.

Referring to the complement pathway, it is part of the innate immune system which, in contrast to the adaptive immune system, does not change over the course of an individual's lifetime. The complement system consists of a number of proteins found in the blood and normally circulating as inactive precursors (zymogens or pro-proteins). Complement system proteins are of high abundance in blood serum and play a role in normal homeostasis. Dysregulated complement activation can thus play a significant role in diseased cancer conditions. Biomarkers involved in the complement pathway include, but are not limited to, a functional sub-population of C1 Inhibitor, also known as Plasma Protease C1 Inhibitor (SERPING1), Complement C1 (C1), Complement (C2), Complement (C3), complement component 4 binding protein alpha (C4BPA), properdin (PROP), Complement C4a (C4a), Complement 5 (C5), and Carboxypeptidase N (CPN1).

Referring to the acute-phase inflammation pathway, inflammatory responses regulate many aspects of tumor development. More specifically, inflammation plays a role throughout tumorigenesis, from initiation all the way to metastatic progression. Acute-phase inflammatory proteins include, but are not limited to, Serum Amyloid 2 (SAA2), extracellular matrix protein 1 (ECM1), chromogranin A (CMGA), Inter-alpha-trypsin inhibitor heavy chain H4 (ITIH4), neutrophil activating peptide-2 (NAP-2), SERPIN proteins (e.g., active/inactive populations of AAT) as well as neutrophil elastase (ELANE).

1.2.3. Prediction Model

In various embodiments, the prediction model, which is executed by a processor of a computer, performs the biomarker level analysis 114 (see FIG. 1A) and outputs a cancer disease state 116 based on the quantitative levels of one or more biomarkers detected by the biomarker quantification assay 112.

In various embodiments, the prediction model is trained using training data that includes quantitative values of biomarker levels obtained from samples derived from patients with known cancer. In various embodiments, the prediction model is trained using training data that includes quantitative values of biomarker levels obtained from samples derived from healthy patients. Therefore, the prediction model is trained to accurately characterize a cancer disease state based on quantitative biomarker values. When the prediction model is deployed (e.g., when the cancer disease state is to be characterized in patient 110), the prediction model is applied to quantitative biomarker values detected by the biomarker quantification assay 112 from a sample obtained from the patient 110 to generate the predicted cancer disease state 116 for the patient 110.

In various embodiments, the prediction model performs multiple evaluations, each evaluation investigating the quantitative levels of one or more biomarkers. For each evaluation, the prediction model assigns an initial score to the evaluation. The prediction model combines the initial scores across different evaluations and outputs a cancer disease state 116 based on the combined initial scores. In various embodiments, the multiple evaluations includes the evaluation of different biomarkers that are involved in one, two, three, or more interconnected, dysregulated pathways (e.g., one or more of complement pathway, acute-phase inflammation pathway, coagulation pathway, or glycolysis pathway).

In various embodiments, evaluating quantitative levels of one or more biomarkers includes determining a ratio between a quantitative level of a biomarker (or a subpopulation of a biomarker) in the sample and a corresponding quantitative level of the biomarker (or the subpopulation of the biomarker) determined in healthy samples. As an example, the determined ratio can be between the quantitative level of the THBS1 biomarker obtained from the sample and the average quantitative level of the THBS1 biomarker determined in healthy samples. Such a determined ratio can be expressed as:

$$Ratio = \frac{{Biomarker}_{sample}}{{Biomarker}_{healthy}}$$

where Biomarkerₛₐₘₚₗₑ refers to the quantitative level of the biomarker determined in the sample and where Biomarker_{healthy} refers to the quantitative level of the biomarker determined in healthy samples.

In some embodiments, evaluating the quantitative level of one or more biomarkers includes determining a ratio between a quantitative level of a subpopulation of a biomarker in the sample and a corresponding quantitative level of the corresponding subpopulation of the biomarker determined in healthy samples. As an example, the determined ratio can be between the quantitative level of a subpopulation of active or inactive SERPIN (e.g., AAT) determined from the sample and the average quantitative level of active or inactive SERPIN (e.g., AAT), respectively, that is determined in healthy samples. Such a determined ratio can be expressed as:

$$Ratio = \frac{Biomarker {Subpopulation}_{sample}}{Biomarker {Subpopulation}_{healthy}}$$

where Biomarker Subpopulationₛₐₘₚₗₑ refers to the quantitative level of the subpopulation of the biomarker determined in the sample and where Biomarker Subpopulation_{healthy} refers to the quantitative level of the subpopulation of the biomarker determined in healthy samples.

In some embodiments, evaluating the quantitative level of one or more biomarkers includes determining an initial ratio between a quantitative level of a first biomarker in the sample and a quantitative level of a second biomarker in the sample. As an example, the determined ratio can be between the quantitative level of ELANE determined from the sample and the quantitative level of a SERPIN (e.g., AAT) in the sample. Such a determined ratio can be expressed as:

$$Initial {Ratio}_{sample} = \frac{First {Biomarker}_{Sample}}{Second {Biomarker}_{Sample}}$$

where First Biomarkerₛₐₘₚₗₑ refers to the quantitative level of the first biomarker determined in the sample and where Second Biomarkerₛₐₘₚₗₑ refers to the quantitative level of the second biomarker determined in the sample.

Next, the predictive model can determine a ratio between the initial ratio in the sample and the corresponding initial ratio determined in healthy samples. An example of this ratio can be expressed as:

$$Ratio = \frac{Initial {Ratio}_{sample}}{Initial {Ratio}_{healthy}}$$

where Initial Ratio_{healthy} further refers to the ratio between the first biomarker determined in healthy samples (e.g., First Biomarker_{healthy}) and the second biomarker determined in healthy samples (e.g., Second Biomarker_{healthy}).

In some embodiments, evaluating the quantitative level of one or more biomarkers includes determining an initial ratio between a quantitative level of a first subpopulation of a biomarker in the sample and a quantitative level of a second subpopulation of a biomarker in the sample. For example, the initial ratio may be between the quantitative level of an inactive subpopulation of SERPIN (e.g., AAT) in the sample and a quantitative level of a second subpopulation of the SERPIN (e.g., AAT). An example of the initial ratio for the sample can be expressed as:

$$Initial {Ratio}_{sample} = \frac{Biomarker First {Subpopulation}_{sample}}{Biomarker Second {Subpopulation}_{sample}}$$

where Biomarker First Subpopulationₛₐₘₚₗₑ refers to the quantitative level of the first subpopulation of the biomarker determined in the sample and where Biomarker Second Subpopulationₛₐₘₚₗₑ refers to the quantitative level of the second subpopulation of the biomarker determined in the sample.

Next, the predictive model can determine a ratio between the initial ratio in the sample and the corresponding initial ratio determined in healthy samples. An example of this ratio can be expressed as:

$$Ratio = \frac{Initial {Ratio}_{sample}}{Initial {Ratio}_{healthy}}$$

where Initial Ratio_{healthy} further refers to the ratio between the first subpopulation of the biomarker determined in healthy samples (e.g., Biomarker First Subpopulation_{healthy}) and the second subpopulation of the biomarker determined in healthy samples (e.g., Biomarker Second Subpopulation_{healthy}).

In some embodiments, evaluating the quantitative level of one or more biomarkers includes determining an initial ratio between a quantitative level of a first biomarker in the sample and a quantitative level of a subpopulation of a second biomarker in the sample. For example, the initial ratio may be between the quantitative level of ELANE in the sample and a quantitative level of a subpopulation of the SERPIN (e.g., AAT) in the sample. An example of the initial ratio for the sample can be expressed as:

$$Initial {Ratio}_{sample} = \frac{First {Biomarker}_{sample}}{Subpopulation of Second {Biomarker}_{sample}}$$

where First Biomarkerₛₐₘₚₗₑ refers to the quantitative level of the first biomarker determined in the sample and where Subpopulation of Second Biomarkerₛₐₘₚₗₑ refers to the quantitative level of the subpopulation of the second biomarker determined in the sample.

Next, the predictive model can determine a ratio between the initial ratio in the sample and the corresponding initial ratio determined in healthy samples. An example of this ratio can be expressed as:

$$Ratio = \frac{Initial {Ratio}_{sample}}{Initial {Ratio}_{healthy}}$$

where Initial Ratio_{healthy} further refers to the ratio between the first subpopulation of the biomarker determined in healthy samples (e.g., First Biomarker healthy) and the second subpopulation of the biomarker determined in healthy samples (e.g., Subpopulation of Second Biomarker_{healthy}).

Altogether, the determined ratio represents a level of dysregulation of biomarker levels in the sample in comparison to biomarker levels in a healthy sample. If biomarkers in the sample are not dysregulated, which may be indicative of a particular cancer state, such as a lack of cancer, the determined ratio is near a value of 1. On the other hand, if biomarkers in the sample are highly dysregulated, which may be indicative of a particular cancer state, such as a presence of cancer, the determined ratio deviates from a value of 1.

The determined ratio can then be compared to a pre-determined threshold ratio and the prediction model can assign an initial score to the evaluation. In one embodiment, the pre-determined threshold value of a biomarker can be derived from training samples that include samples from both cancerous and healthy individuals. Generally, the pre-determined threshold ratio represents a ratio that can be compared to the determined ratio. In other words, if the determined ratio is a ratio between a quantitative level of a biomarker in the sample and the quantitative level of the biomarker in healthy samples, then the pre-determined threshold ratio is a ratio between a quantitative level of the biomarker in cancerous samples and a quantitative level of the biomarker in healthy samples. On the other hand, if the determined ratio is a ratio between an initial ratio of the sample (e.g., between levels of biomarkers in the sample) and an initial ratio of healthy samples (e.g., between levels of biomarkers in healthy samples), then the pre-determined threshold ratio is also a ratio between an initial ratio of levels of biomarkers in cancerous samples and an initial ratio of levels of biomarkers in healthy samples. Therefore, the determined ratio can be compared to the pre-determined threshold ratio.

In one embodiment, the pre-determined threshold value may be expressed as Threshold value=Expected Value+Constant. In one embodiment, the pre-determined threshold value may be expressed as Threshold value=Expected Value−Constant. Here, the Expected value can represent an average of a ratio across healthy and cancer samples in the training data and the Constant can represent a variance of the ratio across healthy and cancer samples in the training data. A variance may further take into account the combined technical and biological variances that may occur across samples.

As one example, if the determined ratio represents a ratio between a level of a biomarker in the sample and the level of the biomarker across healthy samples (e.g.,

$$( e . g . , Ratio = \frac{{Biomarker}_{sample}}{{Biomarker}_{healthy}} ) ,$$

the pre-determined threshold can be any value between 0-10, or more. As specific examples, if the determined ratio is a ratio between the level of any one of THBS1, TIMP1, PPBP, PF4, or CMGA in the sample and the corresponding level of the biomarker in healthy samples, then the pre-determined threshold can be a value of 10. If the determined ratio is a ratio between the level of any one of C3, C4BPA, or PROP in the sample and the corresponding level of the biomarker in healthy samples, then the pre-determined threshold can be a value of 1.5. If the determined ratio is a ratio between the level of any one of SAA2 or ECM1 in the sample and the corresponding level of the biomarker in healthy samples, then the pre-determined threshold can be a value of 1.5. If the determined ratio is a ratio between the level of inactive AAT in the sample and the corresponding level of the inactive AAT in healthy samples, then the pre-determined threshold can be a value of 1.5.

As another example, if the determined ratio is expressed as

$$Ratio = \frac{Initial {Ratio}_{sample}}{Initial {Ratio}_{healthy}} ,$$

where the initial ratio of the sample or healthy is expressed as

$$Initial {Ratio}_{sample} = \frac{Biomarker First {Subpopulation}_{sample}}{Biomarker Second {Subpopulation}_{sample}} ,$$

the pre-determined threshold can be any value between 0-10, or more. As specific examples, if the initial ratio of the sample represents a ratio between an inactive population of AAT and an active population of AAT, the pre-determined threshold value can be 3.5. Therefore, if the ratio is greater than 3.5, then the ratio of inactive to active subpopulations of AAT in the sample is likely dysregulated.

As another example, if the determined ratio is expressed as

$$Ratio = \frac{Initial {Ratio}_{sample}}{Initial {Ratio}_{healthy}} ,$$

where the initial ratio of the sample or healthy is expressed as

$$Initial {Ratio}_{sample} = \frac{First {Biomarker}_{sample}}{Subpopulation of Second {Biomarker}_{sample}} ,$$

the pre-determined threshold can be any value between 0-10, or more. As specific examples, if the initial ratio of the sample represents a ratio between ELANE and an active population of AAT, the pre-determined threshold value can be 10. Therefore, if the ratio is greater than 10, then the ratio of ELANE to the active subpopulation of AAT in the sample is likely dysregulated.

The prediction model compares the determined ratio between the sample and healthy samples to the pre-determined threshold value to determine whether the one or more biomarkers in the sample exhibit beyond a threshold level of dysregulation. In one embodiment, if the determined ratio is greater than a pre-determined threshold value (e.g., greater than Expected Value+Constant) or less than the pre-determined threshold value (e.g., less than Expected Value−Constant), the one or more biomarkers in the sample are dysregulated in comparison to healthy samples.

In one embodiment, the prediction model may assign an initial score for the evaluation based on the comparison between the determined ratio and the pre-determined threshold value. For example, the prediction model may assign an initial score for the evaluation if the determined ratio is greater than or less than the pre-determined threshold value. As another example, the prediction model assigns an initial score that represents a level of dysregulation based on the comparison. For example, the more deviant the determined ratio is from the pre-determined threshold value, the higher the initial score assigned for the evaluation.

In various embodiments, the prediction model combines the initial scores assigned to the individual evaluations and determines a total score for the sample.

In some embodiments, the prediction model assigns weights to each initial score and determines the total score for the sample as the weighted summation of the initial scores. The prediction model may compare the total score for the sample to one or more threshold values to determine a cancer disease state 116. Such a threshold value can be dependent on the type of cancer disease state 116 (e.g., presence/absence of cancer, likelihood of developing cancer, type of cancer, stratification of the patient, etc.). For example, if the cancer disease state 116 is a binary option (e.g., presence/absence of cancer), the prediction model may output a presence of cancer for the patient if the total score for the sample is above the threshold value. Conversely, the prediction model may output an absence of cancer for the patient if the total score for the sample is below the threshold. As another example, if the cancer disease state 116 includes multiple categories (e.g., a likelihood value or a stratification of the patient), the prediction model may compare the total score of the sample to multiple threshold values to determine which category to place the patient in.

1.2.4. Medical Intervention

The pattern of systemic dysregulation through the protein markers disclosed herein could be used as guidance generally for the efficacy for any therapeutic strategy. Threshold variances from this panel can establish signature cancer profiles from blood to support all areas of medical benefit from liquid biopsy technologies. Any therapeutic strategy that can begin to untangle the fibers of this web of dysregulation embedded in these three systemic response pathways may improve the survival of cancer patients. With suitable biomarkers to support a stroma liquid biopsy, accounting for changes in these markers could report whether the network or systemic web of dysregulation is or is not, unwinding back to normalcy.

In various embodiments, the medical intervention that is provided to the patient based on a determined cancer state is a therapeutic agent such as a biologic, e.g. a cytokine, antibody, soluble cytokine receptor, anti-sense oligonucleotide, siRNA, etc. Such biologic therapeutic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists, e.g. IL-1Ra, IL-1 Trap, sIL-4Ra, etc. Also included are any immune modulators (i.e., PD-1 or PDL-1 inhibitors) that redirect the subject's immune system to treat cancer. Also included are biosimilar or bioequivalent drugs to the active agents set forth herein.

In various embodiments, a medical intervention can be an anticoagulant provided to the patient. Anticoagulation therapy may perturb the microenvironment ecosystem, rather than having a direct effect on rapidly proliferating cells. Some patients may be subject to a risk of internal bleeding and therefore, anticoagulant therapy can be deemed appropriate for different patients. For example, anticoagulation therapy may serve as an adjuvant therapy for patients with advanced disease whereas patients with less advanced disease can more readily tolerate anticoagulant therapy.

In various embodiments, biologic therapeutic agents provided to the patient based on a determined cancer state may modulate the activity of proteins involved in interconnected, dysregulated pathways. In one embodiment, a determined cancer state in a patient may reflect hyperactivity of ELANE. Therefore, an inhibitor of ELANE can serve as a biologic therapeutic agent that is administered to the patient. As another example, a determined cancer state in a patient may reflect hyperactivity of a neutrophil enzyme, such as cathepsin G. Cathepsin G cleaves precursor proteins into NAP-2 and therefore, hyperactivity of cathepsin G may be indicated by increased levels of NAP-2. An inhibitor of cathepsin G can serve as a biologic therapeutic agent that is administered to the patient.

In various embodiments, a biologic therapeutic agent administered to the patient can aim to modulate the activity of a dysregulated pathway, such as the complement pathway. For example, a therapeutic agent that inactivates the functionality of the C1 inhibitor can open the gate to the classical complement pathway. In various embodiments, such a therapeutic agent that inactivates the functionality of the C1 inhibitor can be a modulator of the complement pathway. Modulation of the complement pathway has shown therapeutic benefit in chronic pathologies such as macular degeneration and retinal injury. For example, a biologic therapeutic agent can be a substrate that binds to the reactive centre loop (RCL) cleavage site of the C1 inhibitor. In some embodiments, as C1 inhibitor is a pleotropic regulator of both coagulation and complement pathways, indirect inactivation of the C1 inhibitor can also be achieved through the administration of an anti-coagulant biologic therapeutic agent.

A pharmaceutical composition administered to an individual includes an active agent such as the biologic therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method. Such a pharmaceutical composition may be administered for prophylactic (e.g., before determination of a cancer state in the patient) or for treatment (e.g., after determination of a cancer state in the patient) purposes.

In various embodiments, quantitative levels of a panel of biomarkers can be monitored throughout a subject's life, a wellness strategy, so that any changes in the biomarkers, which may point towards risk factors for cancer, can be used for a medical intervention. Hereditary genetic factors that impinge on the functionalities of the regulating SERPIN protease inhibitors in this model of dysregulation may also be viewed in the context of risk factors which can be additional determining factors for medical intervention.

In various embodiments, a medical intervention that is provided to the patient based on a determined cancer state is a suggested lifestyle change such as physical therapy or a change in diet. A medical intervention may additionally support a decision to have, or not to have, surgery or radiation therapy. The method also provides for combination therapy of one or more therapeutic agents and/or suggested lifestyle change, where the combination can provide for additive or synergistic benefits.

### 1.3. Computer Implementation

The methods of the invention, including the step of determining a cancer disease state for a patient through a biomarker level analysis 114, are, in some embodiments, performed on a computer.

For example, the building and execution of a predictive model and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a predictive model of this invention. Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

1.3.1. Example Computer

FIG. 1C illustrates an example computer 180 for implementing the methods depicted in FIGS. 1A and 1B. For example, as described above, the computer 180 can execute code for a prediction model that performs the biomarker level analysis 114, which determines a cancer disease state in a patient. The computer 180 includes at least one processor 102 coupled to a chipset 104. The chipset 104 includes a memory controller hub 120 and an input/output (I/O) controller hub 122. A memory 106 and a graphics adapter 128 are coupled to the memory controller hub 120, and a display 118 is coupled to the graphics adapter 128. A storage device 108, an input interface 124, and network adapter 126 are coupled to the I/O controller hub 122. Other embodiments of the computer 180 have different architectures.

The storage device 108 is a non-transitory computer-readable storage medium such as a hard drive, compact disk read-only memory (CD-ROM), DVD, or a solid-state memory device. The memory 106 holds instructions and data used by the processor 102. The input interface 124 is a touch-screen interface, a mouse, track ball, or other type of pointing device, a keyboard, or some combination thereof, and is used to input data into the computer 180. In some embodiments, the computer 180 may be configured to receive input (e.g., commands) from the input interface 124 via gestures from the user. The graphics adapter 128 displays images and other information on the display 118. The network adapter 126 couples the computer 180 to one or more computer networks. In some embodiments, the computers 180 can lack some of the components described above, such as graphics adapters 128, and displays 118.

The computer 180 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 108, loaded into the memory 106, and executed by the processor 102.

In various embodiments, the prediction model that performs the biomarker level analysis 114 can run on a single computer 180. In some embodiments, the prediction model can be executed across multiple computers 180 communicating with each other through a network such as in a server farm.

### 1.4. Kits

Also disclosed herein are kits for determining a cancer disease state 116. Such kits can include reagents for detecting quantitative levels of one or more biomarkers as well as instructions for performing a biomarker level analysis 114 based on the detected quantitative levels of the one or more biomarkers.

A kit can comprise a set of reagents for generating a dataset via at least one protein detection assay that is associated with a sample from the subject. The dataset can include data representing quantitative levels corresponding to biomarkers described above in Section 1.2.2. In various embodiments, the dataset can include data representing quantitative levels corresponding to two or more biomarkers comprising a first subpopulation of a SERPIN protein and a second subpopulation of a SERPIN protein. In various embodiments, the dataset can include data representing quantitative levels of biomarkers including a total population of AAT, an active subpopulation of AAT, an inactive subpopulation of AAT, and a total population of ELANE. In various embodiments, the dataset can include data representing quantitative levels of biomarkers including two or more of TIMP-1, PPBP, THBS1, PF4, HEP2, C3, C4BPA, PROP, SAA2, a SERPIN protein, ELANE, ECM1 and CMGA.

The instructions included in the kit can be instructions for generating a total score that is indicative of the cancer disease state 116 in the patient 110. Such instructions can include instructions for determining an initial score based on quantitative levels of biomarkers, as is described above in relation to Section 1.2.3, and to mathematically combine the initial scores to generate the total score, wherein a higher total score indicates an increased likelihood of a particular cancer disease state 116, such as a presence of cancer or a higher severity of cancer.

In some embodiments, the reagents included in the kit are reagents for performing LC-MS to quantify the levels of biomarkers. In some embodiments, the reagents included in the kit include the ALBUVOID LC-MS on-Bead reagents. In some embodiments, the reagents include one or more antibodies that bind to one or more of the biomarkers, optionally wherein the antibodies are monoclonal antibodies or polyclonal antibodies. In some embodiments, the reagents can include reagents for performing ELISA including buffers and detection agents.

A kit can further include software for performing instructions included with the kit, optionally wherein the software and instructions are provided together. For example, a kit can include software for executing the predictive model, as is described above in Section 1.2.3. Thus, the software executes the predictive model which mathematically combines quantitative levels of biomarkers generated using the set of reagents.

A kit can include instructions for use of reagents included in the kit. For example, a kit can include instructions for performing at least one protein detection assay such as LC-MS, ALBUVOID LC-MS on-Bead assay, an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from MA, immunofluorescence, immunochemiluminescence, immunoelectrochemiluminescence, immunoelectrophoretic, a competitive immunoassay, and immunoprecipitation.

A kit can include instructions for taking at least one action, such as a medical intervention, based on the determined cancer disease state.

### EXAMPLES

### Example 1: Detecting Biomarker Levels Predictive of a Cancer Disease State

In this report, 3 cancer types—pancreatic, breast and cancer, with samples taken from clinically characterized stages I-IV, were compared against a pooled samples from matched 5 normal/healthy individuals of similar age and sex, in this case females, ages 40-60. Additionally, the variance within these same normal/healthy individuals were considered to account for any combined technical and biological variance with the performed methods.

The workflow for determining biomarker levels in a sample follows the ALBUVOID LC-MS On-Bead sample prep method. In brief, 50 μl serum sample is prepared by adding a binding buffer, then applied to the ALBUVOID beads, and washed. All steps are performed within a microfuge spin-filter format. Albumin is specifically voided out, while the majority of the remaining serum proteome is retained on the bead. Next, On-Bead digestion is conducted for 4 hours to minimize proteolytic background. Specifically, reduction, alkylation and Trypsin digestion all take place on the bead. Peptides are labeled with TMT (isobaric labels). After labeling, the peptides were pooled and analyzed with a single LC-MS/MS 3 hour gradient run using nanoRSLC system interfaced with a THERMO SCIENTIFIC Q EXACTIVE HF (Thermo Scientific) instrument, using data-dependent acquisition with resolution of 60,000, followed by MSMS scans (HCD 30% of collision energy) of 20 most intense ions, with a repeat count of two and dynamic exclusion duration of 60 sec. Normalized thresholds for determining whether certain biomarker levels differed significantly from healthy were established. Specifically, a cancer/healthy ratio for a biomarker that is greater than 1.5 indicates an upregulated biomarker level whereas a cancer/healthy ratio for a biomarker that is less than 0.7 indicates a downregulated biomarker level.

The LC-MS/MS spectral data was searched against the Human Ensembl databases using X!tandem (thegpm.org) with carbamidomethylation on cysteine as fixed modification and oxidation of methionine and deamidation on Asparagine as variable modifications using a 10 ppm precursor ion tolerance and a 20 ppm fragment ion tolerance. The searches were done using an in-House version of X! Tandem with protein filters set based on FPR supplied by the software: valid log(e)<−0.4, p=87, FPR=0.72%. The peptides were filtered by log e<−2 and protein filtered by minimal number of peptide>2.

Example results of various biomarker levels and their respective upregulation/downregulation categorization is shown in Table 1. The column entitled “TMT Report Ratio Threshold” indicates the upregulation or downregulation of a protein biomarker in cancer subjects in relation to healthy subjections.

These results indicate that there is a measurable serum cancer phenotype that can be modeled with categorical proteins taken from: i) inflammation and acute reactants, ii) blood coagulation, iii) tissue remodeling, iv) glycolysis, and v) all others observed here and not previously described for multiple tumors (APOA1, APOC3, TTR, SHBG, SEMA3D, CFAP61, PFKM, SPRED2, C18orf63, CH17-224D4.2, CTD-2007N20.1).

### Example 2: Levels of Biomarkers Involved in Interconnected, Dysregulated Pathways

Normal female human samples (N=4-5) were provided between the ages of 40 and 60, along with cancerous serum samples from females (N=5) within similar age ranges. The diseased samples were as follows: stage 1 breast cancer, stage 2 lung cancer, stage 2b pancreatic cancer, ovarian cancer, and Non-Hodgkin's lymphoma cancer. ALBUVOID LC-MS On-Bead Kit was used to process the samples to deplete albumin.

50 μl of each sample was individually processed with 25 mg of ALBUVOID matrix. The protocol provided in the kit was followed (and samples were eluted with 200 μl of AlbuVoid Elution Buffer). 10 μl of the eluate was loaded for SDS-PAGE, onto NuPage™ 10% Bis-Tris Gel (Invitrogen) and separated for 5 minutes or until all the sample had gone into the gel. Gel was stained with Coomassie Blue R250 and de-stained. The gel piece that has sample (from loading well to running loading dye) was excised and cut into 1 mm cubes. Samples were prepped for in-gel digest after being reduced with 10 mM DTT, incubated at 60 C for 30 min, and alkylated with 20 mM iodoacetamide, incubated at room temperature in the dark for 45 min. After the gel pieces were washed and dried, 1 ug of PIERCE Trypsin Protease was added to digest the proteins overnight in 37° C. The following day, peptides were extracted with 60% acetonitrile, 5% formic acid and dried under vacuum. Samples were re-solubilized to 80 μl in 5% acetonitrile, 0.1% TFA overnight in 4° C. 1 ul was analyzed by LC-MS using Nano LC-MS/MS (Dionex Ultimate 3000 RLSCnano System) interfaced with Q EXACTIVE HF.

Samples were loaded onto a self-packed 100 μm×2 cm trap (Magic C18AQ, 5 μm 200 Å, Michrom Bioresources, Inc.) and washed with loading Buffer A (0.1% trifluoroacetic acid) for 5 min with a flow rate of 10 μl/min. The trap was brought in-line with the analytical column (Magic C18AQ, 3 μm 200 Å, 75 μm×50 cm) and peptides fractionated at 300 nL/min using a segmented linear gradient 4-15% B in 15 min (where A: 0.2% formic acid, and B: 0.16% formic acid, 80% acetonitrile), 15-25% B in 40 min, and 25-50% B in 32 min. Mass spectrometry data was acquired using a data-dependent acquisition procedure with a cyclic series of a full scan acquired in Orbitrap with resolution of 120,000 followed by MS/MS (HCD relative collision energy 27%) of the 20 most intense ions and a dynamic exclusion duration of 20 sec.

The raw data was converted into MASCOT Generic Format (MGF) using Proteome Discover 2.1 (ThermoFisher) and searched against UniProt human database using an in-house version of X!tandem (Global Proteome Machine (GPM) software). For MS based quantitation, the raw data was analyzed using Skyline (Skyline-daily). The Skyline results were filtered so that average mass error (average of chromatogram peaks of a precursor) was below 3 ppm and the isotope dot product (dot product between expected and observed precursor isotope distribution) was greater than 0.9. Spectral counts report any peptide observable and annotated to the protein identification.

Quantified levels of different biomarkers involved in different interconnected pathways in healthy and cancer patients are shown in Table 2. In particular, the biomarkers shown in Table 2 are involved in one or more of the coagulation pathway, complement pathway, acute-phase inflammation pathway and other pathways (fibrinolysis and pregnancy associated pathways). Of interest are levels of THBS1, TIMP1, PPBP, PF4, CMGA, and SAA2, which are each elevated in cancer at least 10 times their corresponding levels in healthy individuals. Additionally of interest are the levels of C3, C4BPA, PROP, and an active subpopulation of HEP2, which are each lower in cancer by at least 1.5 times in comparison to their corresponding levels in healthy individuals. Additionally of interest is the level of ECM1, which is elevated in cancer at least 1.5 times in comparison to their corresponding levels in healthy individuals.

Additionally, quantified levels of various SERPIN proteins in healthy and cancer patients are shown in Table 3. Generally, in Tables 2 and 3, upregulated and downregulated biomarkers in the different cancers in relation to healthy subjects are indicated by upward and downward arrows.

### Example 3: Cancer Serum Phenotype Involving AAT and/or ELANE Biomarkers

Samples taken from cancer types including pancreatic, breast, ovarian, Non-Hodgkin's Lymphoma, and lung cancer were compared against a pooled sera from matched normal/healthy individuals of similar age and sex, in this case females (N=4), ages 40-60. Additionally, the comparison also considered the variance within these same normal/healthy individuals to account for any combined technical and biological variance.

### Example 3.1: Determining Subpopulations of AAT

Proteolytic activity refers to processes that degrade proteins, and the family of proteins that perform this activity are called proteases. Proteolytic events, unlike those in most chemical and biochemical reactions, do not follow ideal reaction conditions in which the final products are formed through equilibria within and between the relative concentrations of the reactants and products. This is because in proteolysis, the key reactant is water (hydrolysis) and thus the reaction is unidirectional, as water molecules are in virtually infinite supply and cannot be exhausted. Thus, organisms have evolved a complex system of regulation that allows for multiple factors, both macromolecules and small molecules, to control aberrant proteolytic events. In blood, these regulating events are often overlapping with multiple pathways and regulating mechanisms (i.e., protease inhibitors), all subject to insults that can perturb the delicate balance of the proteolytic web, causing chronic pathologies.

The suicidal serine protease inhibitors (SERPIN) are a collection of a super-family of proteins annotated within the SERPIN gene nomenclature. These proteinase inhibitors regulate key intracellular and extracellular pathways. Some representative examples of SERPINs among the key regulators in blood serum, include SERPINA1 (also known as AAT), which protects lung tissue from ELANE. SERPINs differ from all other families of protease inhibitors in having a complex mechanism of action that involves a drastic change in their shape, forming the basis of a suicidal substrate inhibition mechanism. An amino acid region forms the reactive centre loop (RCL) extending out from the body of the protein and directs binding to the target protease. The protease cleaves the SERPIN at the reactive bond site within the RCL, establishing a covalent linkage between the carboxyl group of the SERPIN reactive site and the serine hydroxyl of the protease. The resulting inactive serpin-protease complex is highly stable, and the structural disorder induces its proteolytic inactivation. As a consequence, the protease is permanently inhibited and functionally inactivated.

A cancer serum phenotype can be characterized by measuring AAT proteoforms separated at the protein level, to have the majority of the peptide region RCL-cleaved reporting sub-population in one fraction, and the remaining sub-population, containing the peptide region, in part the RCL-intact sub-population in a second fraction, so as to report these minimum of 2 fractions, at peptide and functional levels, to distinguish sub-population signatures of AAT in cancer sera.

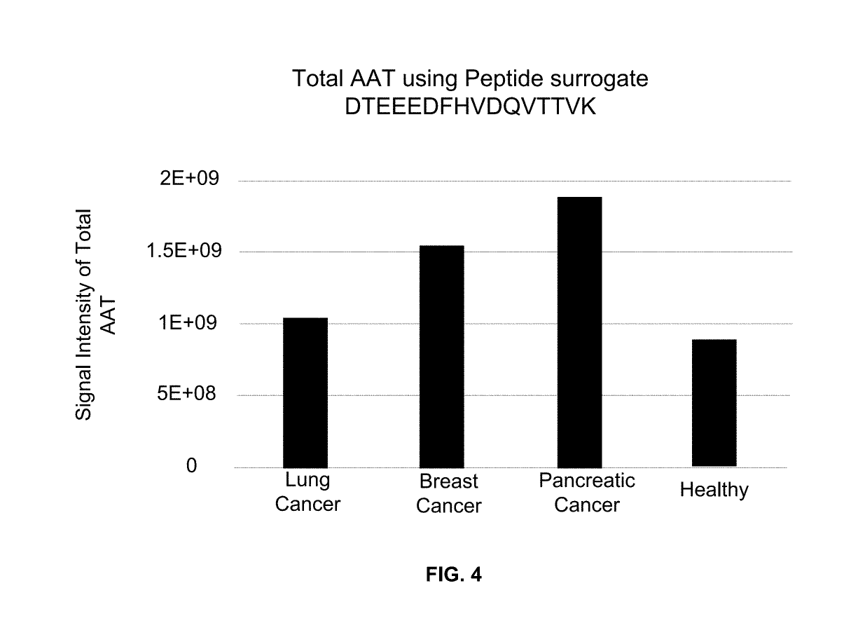

Table 4 documents different subpopulations and total populations of AAT detected in samples that are analyzed through the ALBUVOID on-bead methods described above in relation to Example 1. The “Bead Bound” subpopulation of proteins shown in FIG. 4 refers to proteins that bind to the bead. The “Flow-Through” subpopulation of proteins shown in FIG. 4 refers to proteins that flow through and remain unbound from the ALBUVOID beads. The “Serum Untreated” population refers to the total population of proteins that are observable in serum without any sample processing, that is without ALBUVOID.

In particular, Table 4 reports the normalized ratio of the subpopulations AAT determined from samples obtained from pancreatic cancer patients in comparison to the corresponding subpopulations of AAT in healthy subjects. Specifically, the TMT ratio depicted in Table 4 refers to the ratio between levels detected in pancreatic cancer samples and the levels detected in healthy samples. Table 4 depicts the adjacent RCL tryptic peptide at Lys367 (which is adjacent to the amino acid region of the RCL between 368-392). Additionally, Table 4 documents the RCL intact peptide, a Trypsin truncated amino acid sequence of the full RCL sequence -GTEAABAMFLEAIPMSIPPEVKFNK. Furthermore, Table 4 documents two RCL peptides that are cleaved at Met382 due to suicidal substrate interaction.

These data suggest that the overall AAT population is dominated by the inactive sub-population of AAT collected in the flow-through fraction (e.g., unbound), and this same inactive sub-population dominates the analysis when untreated sera is investigated. These methods distinguish the inactive sub-population of AAT that is not bound to the bead and the active sub-population of AAT that is bound to the bead. When measured as a ratio of these two sub-populations, the ratio may serve as a distinguishable pattern of early dysregulation observable in the cancer serum phenotype. For this analysis, the ratio of the Adjacent RCL Tryptic peptide region would be 1.78/0.35=5.

### Example 3.2: Repeated Verification of the Determined Subpopulations of AAT

A second series of label tests were conducted to verify the first tests. Specifically, this second test followed the same workflow and all 3 pooled cancer sera similarly reported a down-regulated RCL-intact active AAT subpopulation.

The workflow follows the ALBUVOID LC-MS On-Bead sample prep method. In brief, 50 μl serum is prepared by adding a binding buffer, then applied to the ALBUVOID beads, and washed. All steps are performed within a microfuge spin-filter format. Albumin is most especially but not solely voided out, while the majority of the remaining serum proteome is retained on the bead. After the final wash, reduction, alkylation and Trypsin digestion all take place on the bead. After labeling, the peptides were pooled and analyzed with a single LC-MS/MS 3 hour gradient run using nanoRSLC system interfaced with a THERMO SCIENTIFIC Q EXACTIVE HF (Thermo Scientific) instrument, using data-dependent acquisition with resolution of 60,000, followed by MSMS scans (HCD 30% of collision energy) of 20 most intense ions, with a repeat count of two and dynamic exclusion duration of 60 sec.

The LC-MS/MS spectral data was searched against the Human Ensembl databases using X!tandem (thegpm.org) with carbamidomethylation on cysteine as fixed modification and oxidation of methionine and deamidation on Asparagine as variable modifications using a 10 ppm precursor ion tolerance and a 20 ppm fragment ion tolerance. The searches were done using the Rutgers Proteomics Center in-House version of X! Tandem with protein filters set based on FPR supplied by the software: valid log(e)<−0.4, p=87, FPR=0.72%. The peptides were filtered by log e<−2 and protein filtered by minimal number of peptide>2.

The workflow considered:
Normal female ages 40-60 (n=5):
Breast Stage 1 cancer female ages 40-60 (n=2)
Pancreatic Stage 1 cancer female ages 40-60 (n=3)
Lung Stage 2 cancer female age 40-60 (n=1)
Tests used the ALBUVOID LC-MS On-bead protocol
On-Bead Digestion, 4 hours to minimize proteolytic background
Single 3 hour LC-MS, no peptide level fractionation
TMT (isobaric) labels, ratio cancer/normal thresholds >1.5 up-regulated, <0.7 down-regulated
>200 Proteins observed

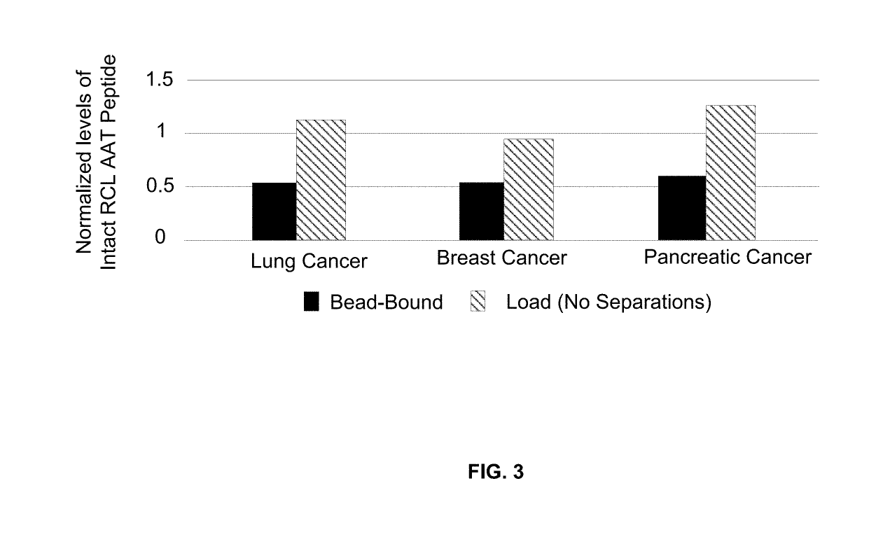

Results are shown in FIG. 3, which confirm the determined subpopulations of AAT that were observed in Example 3.1. These results suggest that the Bead-bound fraction (e.g., RCL-intact, active sub-population of AAT) in the different cancers, using on-bead 4 hour digests, is downregulated relative to the same in pooled normal/healthy controls. Additionally, the total protein load in each of the cancer samples is relatively similar to the total protein load relative to the same in pooled normal/healthy controls.

### Example 3.3: Further Validation of the Determined Subpopulations of AAT

To determine if the methods introduced in Examples 3.1 and 3.2 introduced analytical bias, a label-free analysis of the same basic workflow was performed using a targeted quantification strategy focused on only Alpha-1-Antitrypsin and Neutrophil Elastase.
Serum sample sets, pooled as follows:
Normal female ages 40-60 (n=5):
Breast Stage 1 cancer female ages 40-60 (n=2)
Pancreatic Stage 1 cancer female ages 40-60 (n=3)
Lung Stage 2 cancer female age 40-60 (n=1)
Tests used the ALBUVOID LC-MS On-bead protocol

Briefly, 50 μl of serum was diluted in 100 μl of ALBUVOID LC-MS Kit buffer “AVBB” and added to 25 mg of ALBUVOID beads. Sample was vortexed and centrifuged. Filtrate is collected as Flow-Through (FT).

Samples washed 3× with 250 μl of ALBUVOID LC-MS Kit buffer “AVWB”. (Filtrate combined and saved at 80° C.). For on-bead digest tests, the ALBUVOID LC-MS On-bead protocol (commercial product supplied by Biotech Support Group LLC, Monmouth Junction N.J.) was used. Briefly, 10 mM of DTT in ALBUVOID LC-MS Kit buffer “AVWB” was added to bead and vortexed for 10 minutes and incubated for 30 minutes at 60 C. After samples cool to RT 20 mM Iodoacetamide (in ALBUVOID LC-MS Kit buffer “AVWB”) was added and incubated in the dark for 45 minutes. Samples centrifuged and filtrate was discarded. Bottom of spin-X tubes were rinsed with 50% ACN, in ALBUVOID LC-MS Kit buffer “AVWB” twice. 16 ug/200 ul of trypsin in ALBUVOID LC-MS Kit buffer “AVWB” was added to bead and kept in warm room for 4 hours. (32 μl of 0.5 μg/μl trypsin in 168 μl ALBUVOID LC-MS Kit buffer “AVWB”). Samples were centrifuged and filtrate was collected. 150 μl of 10% formic acid in 50 mM HEPES was added to extract further peptides, vortexed for 10 minutes and centrifuged. Filtrate was combined. Assuming there is ˜800-1000 μg of protein after ALBUVOID processing, there is ˜3 μg/μl of protein in the filtrate. Took 2 μl diluted to 60 μl with water. Samples were loaded 5 μl onto nanoRSLC system interfaced with a THERMO SCIENTIFIC Q EXACTIVE HF (Thermo Scientific) instrument, (0.5 μg) using 2 hour gradient with target.

For In-gel digest of total serum without any separations and defined as “Load” or “Total”, Flow-through (“FT”) is defined as the sub-population of proteins not bound to the ALBUVOID beads, and Elution (“El”), proteins that bound to the ALBUVOID beads but releases using a suitable eluent buffer, for all four samples. In brief, the protocol used an amount of protein after ALBUVOID processing of ˜1000 μg. According to the gel electrophoresis there was estimated 200 μg protein in Load/Total, 100 μg in FT, 40 μg in elution (Elution has the least amount of protein in the gel (1000/25). After reduction/alkylation by common methods known to those in the art, for digestion, Trypsin was added with a 1:50 enzyme:protein ratio. Load serum samples got 4 μg trypsin, FT samples got 2 μg, and Elution got 0.5 μg. After second precipitation samples were solubilized and loaded as such: Total: Solubilized in 100 μl to become 2 μg/μl. Then 1 μl protein diluted to 20 μl to become 0.1 μg. 0.5 μg was loaded (5 μl), FT: Solubilized in 100 μl to become 1 μg/μl. Then 1 μl protein diluted to 10 μl to become 0.1 μg. 0.5 μg was loaded (5 μl), Elution: Solubilized in 80 μl to become 0.5 μg/μl. 0.5 μg was loaded (1 μl). Samples were loaded onto nanoRSLC system interfaced with a Thermo Scientific Q EXACTIVE HF (Thermo Scientific) instrument, using 2 hour gradient with target.

Peptides were solubilized in 0.1% trifluoroacetic acid, and analyzed by Nano LC-MS/MS (Dionex Ultimate 3000 RLSCnano System) interfaced with QExactive HF. Results are shown in Table 2. Samples were loaded onto a self-packed 100 μm×2 cm trap (Magic C18AQ, 5 μm 200 Å, Michrom Bioresources, Inc.) and washed with loading Buffer A (0.1% trifluoroacetic acid) for 5 min with a flow rate of 10 μl/min. The trap was brought in-line with the analytical column (Magic C18AQ, 3 μm 200 Å, 75 μm×50 cm) and peptides fractionated at 300 nL/min using a segmented linear gradient 4-15% B (A: 0.2% formic acid, B: 0.16% formic acid, 80% acetonitrile) in 15 min, 15-25% B in 40 min, 25-50% B in 32 min. Mass spectrometry data was acquired using a MRM procedure that target the 2+ or 3+ of tabularized peptides through-out the run.

The MSMS related parameters were set as following: MSMS resolution: 30K, AGC target: 5E5, isolation window+/−0.7 dalton, normalized collision energy 27, data was recorded in centroid mode.

Raw data analyzed using Thermo Xcalibur (Thermo fisher). Most peptides were analyzed automatically and inspected manually in quanbrowser. Some peptides were manually integrated in qualbrowser due to poor peak shape. 3-4 transitions were used to quantify each peptide. The following peptides were observed and quantified as the totality of signal intensities computationally by common methods known in the art of SRM/MRM target quantifications.

To compare profiles of cancer sera within a proteomics context, total AAT was determined using a common surrogate peptide unrelated to the AAT RCL region (amino acid sequence DTEEEDFHVDQVTTVK). Using this peptide as surrogate or proxy for the total amount of AAT present in the sera, a similar pattern of up-regulation of the total AAT is observed, as is depicted in FIG. 4.

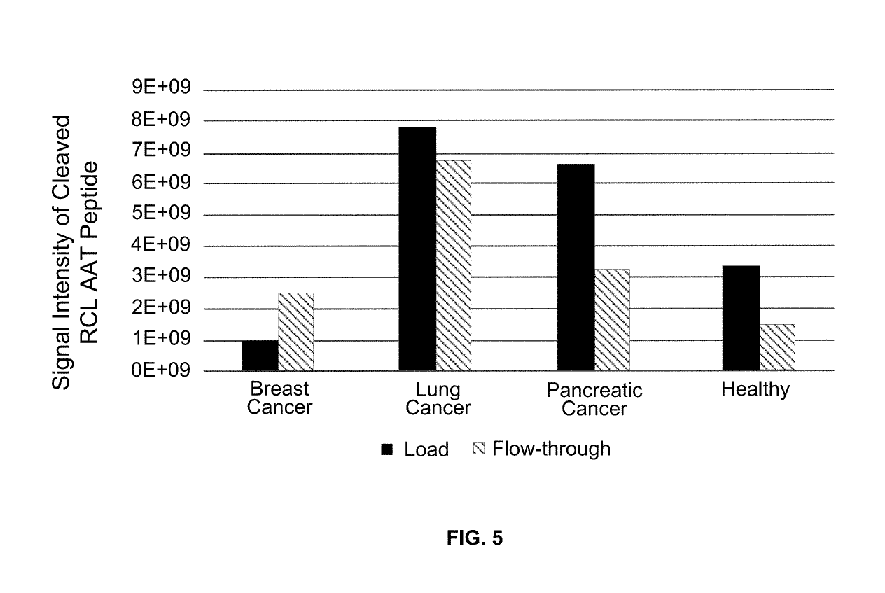

However, with protein level separation (e.g., flow-through shown in FIG. 5)—in this case using ALBUVOID, a more distinctive pattern of AAT was observed. This supports that there are two distinct proteoform sub-populations of AAT, each sub-population having more or less binding bias to the ALBUVOID beads.

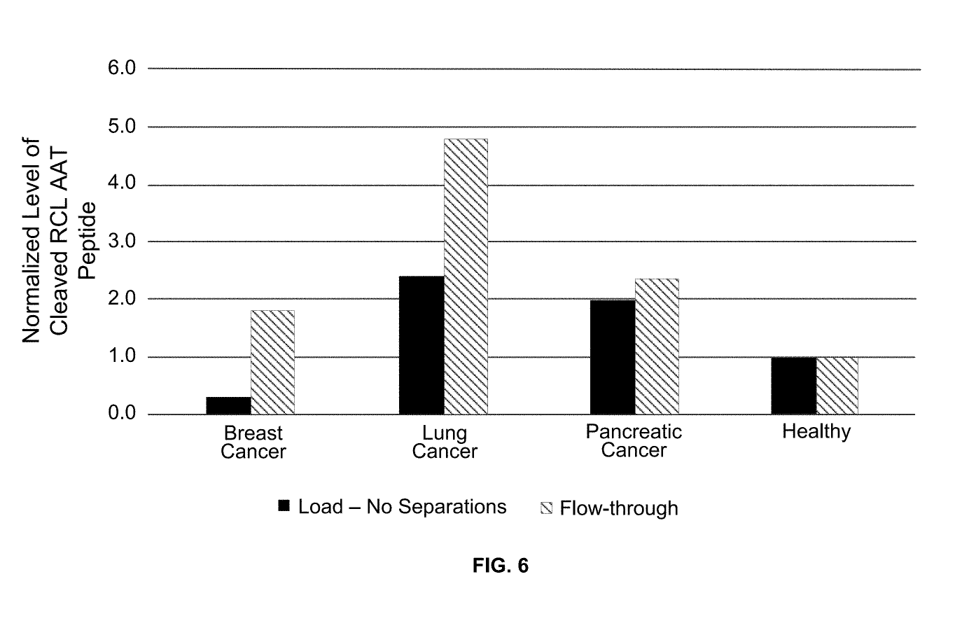

While the overall abundance as reported here support higher amounts of the cleaved RCL peptide reporting feature (presumed to be inactive AAT) in 2 out of the 3 cancer (with Breast the exception), with protein level separation, the flow-through sub-proteome reflects a strong relationship between up-regulated cleaved RCL peptide compared to normal. When these ratios are compared and normalized to the normal sample (normal/normal being=1), the normalized flow-through ratios indicate between a 1.5 times (e.g., breast cancer) to a 5 times (e.g., lung cancer) increase. The cleaved RCL peptide proteoform has a negative binding bias as it poorly binds to the beads, as depicted in FIG. 6. This supports a preferred method that protein level enrichment may be needed to establish protein biomarkers for quantitatively measuring an inactive component of AAT, as being dysregulated in serum/plasma with incidence of cancer.

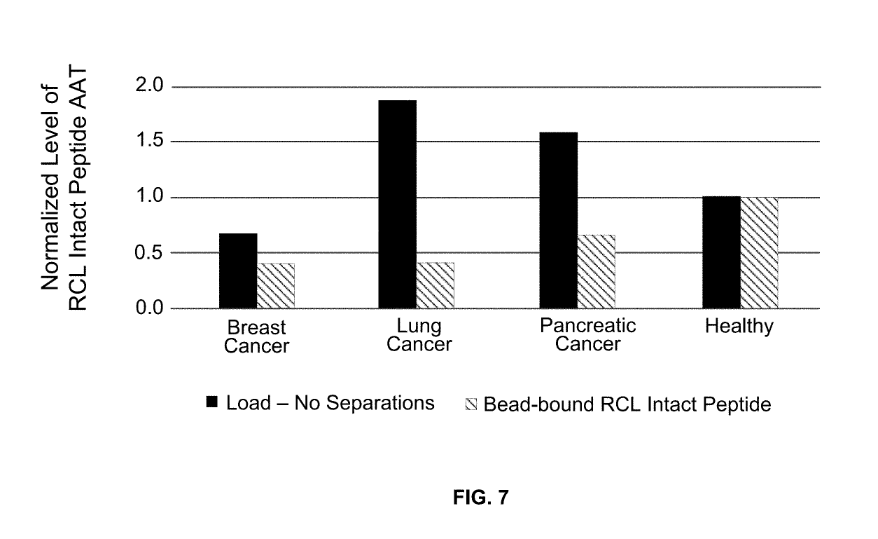

The RCL intact peptide serves as a proteoform feature that distinguishes the maintenance of inhibitory capacity to that which does not. Though the RCL-Intact proteoform also reports in the flow-through (unbound) fraction of the ALBUVOID beads, the proteoform with bias towards binding to ALBUVOID beads are particularly distinguishable as a biomarker for cancer. The bead-bound fraction has a lower amount for all three cancers when normalized to the normal/healthy controls, as depicted in FIG. 7. Specifically, the value of the bead bound RCL intact peptide (e.g., active subpopulation AAT) for each of breast cancer, lung cancer, and pancreatic cancer is 0.7 times or less relative to the corresponding value of the bead bound RCL intact peptide from healthy samples. This decrease in the active subpopulation of AAT is further emphasized in lung cancer and pancreatic cancer as the normalized total AAT (e.g., load—no separations in FIG. 7) is higher in cancer in comparison to healthy.

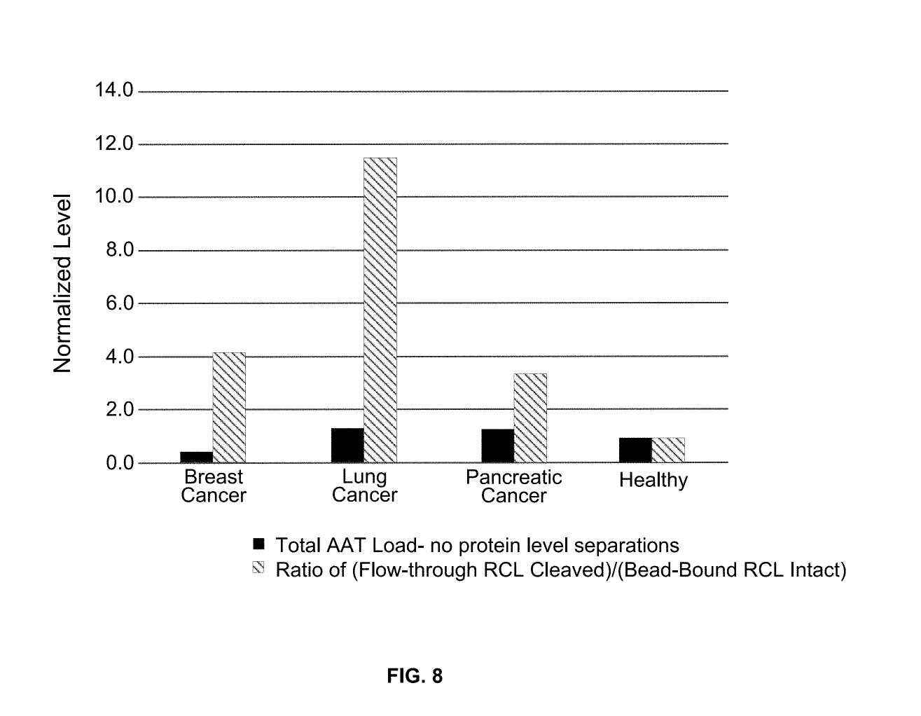

FIG. 8 further depicts the normalized total AAT load and normalized ratio of inactive AAT to active AAT in patients with breast, lung, or pancreatic cancer. FIG. 8 suggests that there is a distinguishable biomarker basis for cancer monitoring based on the ratio of inactive AAT to active AAT. Specifically, for each of breast cancer, lung cancer, and pancreatic cancer, the ratio between the inactive subpopulation of AAT (flow through RCL cleaved) and the active subpopulation of AAT (bead bound RCL intact) is at least 3.5 times greater than the corresponding ratio from healthy samples. As part of the aforementioned SERPIN protein family of suicidal inhibitors, AAT is a protein to which an irreversible conformational switch kills its inhibitory capacity. So even though the total AAT load may increase, as is the case for lung cancer and pancreatic cancer in FIG. 8, the normalized ratio of inactive protein variant (reported as a RCL-intact proteoform separated and bound to a protein-binding bead) to the active RCL-cleaved proteoform increases with significantly more variance.

This evidence suggests that the active AAT subpopulation, which acts as a regulatory gatekeeper of elastase proteolysis, is nearly or completely exhausted. Therefore, as a consequence of active AAT exhaustion and due to this imbalance, the cancer likely maintains uncontrolled elastase activity which can be monitored not from the tumor localized microenvironment, but rather directly from blood serum or plasma.

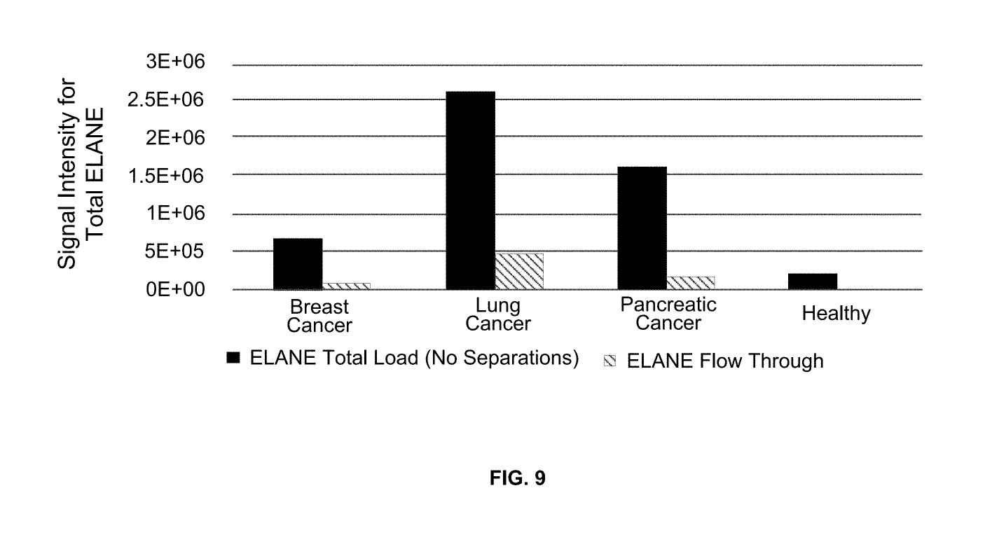

Finally, FIG. 9 depicts severe up-regulation of NE in the cancer sera with no dependence on complexation with AAT. These results support direct measurement data concluding that the same tissue localized dysregulation of NE in cancer can be suggestive for predicting cancer disease state.

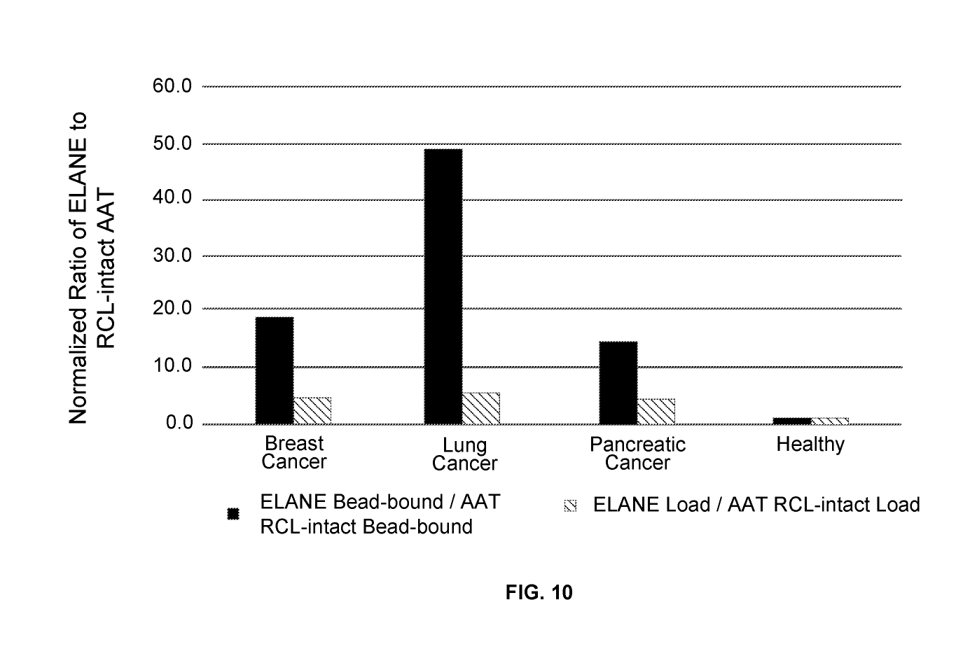

Taken together these observations support a gross systemic imbalance between NE activity and its primary inhibitor—the functional and active form of AAT. This can be reported as a ratio, of NE to ACTIVE AAT. By way of demonstrating this, FIG. 10 depicts the ratio of ALBUVOID bead-bound NE cancer signal intensities divided by the normal signal intensities (defined as NE Bead-bound Normalized) vs. ALBUVOID Bead-bound AAT RCL-intact cancer signal intensities divided by the normal signal intensities (defined as AAT RCL-intact Bead-bound Normalized). Here, the ratio (e.g., NE bead-bound/active AAT subpopulation) for each of the cancers (e.g., breast cancer, lung cancer, and pancreatic cancer) is greater than 10 times the corresponding ratio from the healthy sample.

### Example 3.4: Subpopulations of AAT in Additional Cancers

The quantitative levels of the active and inactive subpopulations of AAT in various cancers (e.g., breast, lung, pancreatic, ovarian, and non-Hodgkin's lymphoma) were additionally evaluated according to the methods described above in Example 2.

Quantified levels of inactive AAT and active AAT in healthy and cancer patients are shown in Table 5. In particular, the subpopulation of inactive AAT in healthy patients is between 4.7 and 12.2 μg/mL whereas the subpopulation of inactive AAT in cancerous patients is broader between 4.7 and 28.4 μg/mL. Additionally, the subpopulation of active AAT in healthy patients is between 1.5 and 10.8 μg/mL whereas the subpopulation of active AAT in cancerous patients is significantly lowered (e.g., between 0.1 and 4 μg/mL) for each respective cancer. Thus, this leads to a ratio of inactive AAT to active AAT in cancerous patients that ranges from 7.1 up to 45.7. This is significantly higher than the ratio of inactive AAT to active AAT in healthy patients that ranges from 0.9 up to 3.2. The significant change in the ratio is at least a reflection of the reduced active subpopulation of AAT in cancerous patients in comparison to the active subpopulation of AAT in healthy patients. As such, the ratio of inactive AAT to active AAT in samples obtained from a cancerous patient can be informative for determining a state of the cancer in the patient.

To summarize, these results demonstrate actionable dysregulated pathways involved in cancer that can be therapeutically targeted to improve cancer patient outcomes. Monitoring biomarker levels involved in the dysregulated pathways can be informative for determining a cancer state without regard to the primary tumor site or progressive stage of clinical disease. The determined cancer state can then be applied towards early detection, diagnostics, prognostics and precision therapeutic modulation.

While embodiments of the invention has been particularly shown and described with reference to a preferred embodiment and various alternate embodiments, it will be understood by persons skilled in the relevant art that various changes in form and details can be made therein without departing from the spirit and scope of the invention.

### TABLES

TABLE 1
Changes in quantitative levels of biomarkers in different cancer samples
in relation to the corresponding levels of biomarkers in healthy samples

| | | Plasma | | | | |
|---|---|---|---|---|---|---|
| | | Proteome | | TMT | | |
| | | Annotation | | Report | | |
| | | (approx. | | Ratio | # Individuals | |
| Protein ID | Protein Description | conc.) | | Threshold | Threshold/All | Cancer Types |
| ITIH3/4 | Inter alpha trypsin | 40 | μg/ml | >1.5 | 7/13 | Pancreatic, |
| (at least | inhibitor, heavy chain | | | (up- | | Breast, |
| one | 3/4 | | | regulated) | | Lung |
| subunit | | | | | | All Stages |
| chain) | | | | | | |
| APOA1 | Apolipoprotein A I | 1 | mg/ml | <0.7 | 9/13 | Pancreatic, |
| | | | | (down- | | Breast, |
| | | | | regulated) | | Lung |
| | | | | | | All Stages |
| APOC3 | Apolipoprotein C III | 100-200 | μg/ml | <0.7 | 5/13 | Pancreatic, |
| | | | | (down- | With APOA1 | Breast, |
| | | | | regulated) | 11/13 | Lung |
| | | | | | | 4/5 Stage 1 |
| CRP | C-reactive protein | 1-2 | μg/ml | >1.5 | 7/11 | Pancreatic, |
| | | | | (up- | | Breast, |
| | | | | regulated) | | Lung |
| | | | | | | All Stages |
| PF4 | Platelet Factor 4 | 5-10 | ng/ml | >1.5 | 13/13 | Pancreatic, |
| | | | | >2.0 | 9/13 | Breast, |
| | | | | (up- | | Lung |
| | | | | regulated) | | All Stages |
| PPBP | Beta | 5-10 | μg/ml | >1.5 | 13/13 | Pancreatic, |
| | thromboglobulinaka | | | >2.0 | 9/13 | Breast, |
| | CTAPIII/NAP-2 | | | (up- | | Lung |
| | | | | regulated) | | All Stages |
| CLU | Clusterin | 100 | μg/ml | >1.5 | 12/13 | Pancreatic, |
| | | | | (up- | | Breast, |
| | | | | regulated) | | Lung |
| | | | | | | All Stages |
| PIGR | Polymeric | 25 | ng/ml | >1.5 | 3/5 | Pancreatic, |
| | immunoglobulin | | | (up- | | All Stages |
| | receptor | | | regulated) | | |
| TTR | Transthyretin | 100-400 | μg/ml | <0.5 | 4/5 | Pancreatic, |
| | | | | (down- | | All Stages |
| | | | | regulated) | | |
| THBS1 | Thrombospondin I | 200 | ng/ml | >1.5 | 3/3 | Lung, All |
| | | | | (up- | | Stages |
| | | | | regulated) | | |
| SAA2 | Serum amyloid A2 | 5 | μg/ml | =10 | 5/5 | Pancreatic, |
| | | | | 1 spectrum, | | Breast, |
| | | | | 2 tests, | | Stage 1 |
| | | | | quantitation | | |
| | | | | may be | | |
| | | | | misleading | | |
| AGT | Angiotensinogen | 40-60 | μg/ml | <0.7 | 5/5 | Pancreatic, |
| | Aka SERPINA8 | | | | | All Stages |
| SERPING1 | serpin peptidase | Common | | >1.5 | 12/13 | Pancreatic, |
| | inhibitor, clade G (C1 | peptides in | | (up- | | Breast, |
| | inhibitor), member 1 | Peptide Atlas | | regulated) | | Lung |
| | | | | | | All Stages |
| CPN1 | Carboxypeptidase N, | 35 | μg/ml | >1.5 | 10/13 | Pancreatic, |
| | serum | | | (up- | | Breast, |
| | | | | regulated) | | Lung |
| | | | | | | All Stages |
| IGLV3-27 | Ig-like | Common | | <0.7 | 9/10 | Pancreatic, |
| | | peptides in | | (down- | | Breast, |
| | | Peptide Atlas | | regulated) | | Lung |
| | | | | | | 5/5 Stage 1 |
| IGHV1OR15-1 | Ig-like | Common | | <0.7 | 2/3 | Lung |
| | | peptides in | | (down- | | All Stages |
| | | Peptide Atlas | | regulated) | | |
| IGHV3-53 | Ig-like | Common | | <0.7 | 3/3 | Breast, |
| | | peptides in | | (down- | | Lung |
| | | Peptide Atlas | | regulated) | | Stages 1 |
| IGKV1D-33 | Ig-like | Common | | >2.0 | 3/3 | Breast, |
| | | peptides in | | (up- | | Lung |
| | | Peptide Atlas | | regulated) | | Stages 1 |
| SHBG | Sex hormone binding | 100-200 | ng/ml | >1.5 | 5/5 | Pancreatic, |
| | globulin | | | (up- | | All Stages |
| | | | | regulated) | | |
| SEMA3D | Sema domain | Annotated to | | <0.7 | 3/3 | Lung |
| | immunoglobulin | Plasma | | (down- | | All Stages |
| | secreted 3D | Proteome, no | | regulated) | | |
| | | concentration | | | | |
| | | data | | | | |
| C4BPB | Complement | 200-500 | μg/ml | >1.5 | 4/5 | Pancreatic, |
| | component 4 binding | | | (up- | | All Stages |
| | protein, beta | | | regulated) | | |

| | | | | | |
|---|---|---|---|---|---|
| CFAP61 | Cilia- and flagella- | Observed | <0.5 | 7/8 | Pancreatic, |
| | associated protein 61 | tissue only, | (down- | | Breast, |
| | | not observed | regulated) | | All Stages |
| | | in Peptide | | | |
| | | Atlas for | | | |
| | | Plasma | | | |
| PFKM | Phosphofructokinase 1 | Cytosolic | >1.5 | 5/5 | Pancreatic, |
| | | protein, few | (up- | 3/5 | Breast, |
| | | observations | regulated) | | Stage 1 |
| | | in Peptide | >2.0 | | |
| | | Atlas for | (up- | | |
| | | Plasma | regulated) | | |
| SPRED2 | Sprouty related EVH1 | Observed | >2.0 | 4/5 | Pancreatic, |
| | domain containing 2 | tissue only, | (up- | 3/5 | Breast, |
| | | not observed | regulated) | | Stage 1 |
| | | in Peptide | >4.0 | | |
| | | Atlas for | (up- | | |
| | | Plasma | regulated) | | |
| C18orf63 | No annotation, simply | Only 2 tissue | >5.0 | Inconsistently | Pancreatic, |
| | called Uncharacterized | observations | (up- | observed | Breast, |
| | | in Peptide | regulated) | | Lung |
| | | Atlas | | | All Stages |
| CH17-224D4.2 | No annotation | Not observed | >1.5 | 3/5 | Pancreatic, |
| | | in Peptide | (up- | | All Stages |
| | | Atlas, No | regulated) | | |
| | | annotation in | | | |
| | | Uniprot | | | |
| CTD-2007N20.1 | No annotation | Not observed | <0.5 | 6/8 | Pancreatic, |
| | | in Peptide | (down- | | Breast, |
| | | Atlas, No | regulated) | | All Stages |
| | | annotation in | | | |
| | | Uniprot | | | |

TABLE 2
Quantitative levels of biomarkers from interconnected pathways obtained from healthy and cancerous samples.

| Protein Concentration | | | | | | Cancer | | | | |
|---|---|---|---|---|---|---|---|---|---|---|
| Range >5 log measurable | | Healthy | | | | Stage 1 | Stage 2 | Non- | Stage 2b | |
| in 1 LC-MS Analysis | | Subject | Subject | Subject | Subject | Breast | Lung | Hodgkin's | Pancreatic | Ovarian |
| Systemic Pathway | Gene Identifier or Protein Code | 1 | 2 | 3 | 4 | Cancer | Cancer | Lymphoma | Cancer | Cancer |
| Coagulation | Tissue Inhibitor of | ND | 0.3 | 0.4 | ND | 0.8↑ | 3.2↑ | 4.0↑ | 1.0↑ | 2.5↑ |
| | Metalloproteinases 1 (TIMP1) | | | | | | | | | |
| Coagulation | Pro-platelet Basic Protein | 5.0 | 3.0 | 2.0 | 3.0 | 107↑ | 201↑ | 26↑ | 80↑ | 15↑ |
| | (PPBP) | | | | | | | | | |
| Coagulation | Platelet Factor 4 (PF4) | ND | ND | ND | ND | 39↑ | 87↑ | 11↑ | 22↑ | 11↑ |
| Coagulation | Thrombospondin 1 (THBS1) | 0.1 | ND | ND | ND | 7↑ | 11↑ | 2↑ | 4↑ | 1↑ |
| Coagulation | Heparin Cofactor II | 1.0 | 1.4 | 0.9 | 1.5 | 0.6↓ | 0.5↓ | 0.3↓ | 0.2↓ | 0.2↓ |
| | (SERPIND1) | | | | | | | | | |
| Coagulation | Extracellular Matrix Protein 1 | 3.0 | 4.0 | 7.0 | 7.0 | 9.0 | 11↑ | 59↑ | 9.0 | 20↑ |
| | (ECM1) | | | | | | | | | |
| Complement | Complement Component 3 (C3) | 1.8 | 1.3 | 1.6 | 1.3 | 1.2 | 0.5↓ | 1.1 | 0.5↓ | 0.5↓ |
| Complement | C4b-binding protein alpha chain | 3.1 | 1.2 | 1.5 | 2.6 | 0.8↓ | 3.2 | 0.6↓ | 0.8↓ | 0.2↓ |
| | (C4BPA) | | | | | | | | | |
| Complement | Properdin (PROP) | 2.5 | 1.6 | 1.3 | 2.3 | 1.2 | 1.5 | 0.6↓ | 0.8↓ | 0.4↓ |
| Complement | Complement Factor P (CFP) | 14.0 | 10.0 | 15.0 | 10.0 | 11.0 | 9.0 | 7.0 | 6.0 | 3.0 |
| Acute-phase | Serum Amyloid A2 (SAA2) | 0.5 | 0.5 | ND | 0.4 | 0.8↑ | 15.2↑ | 4.8↑ | 4.9↑ | 1↑ |
| Inflammation | | | | | | | | | | |
| Acute-phase | Chromogranin-A (CMGA) | | | | | | | 28.0 | | |
| Inflammation | | | | | | | | | | |
| Acute-phase | Ratio: AAT/[AAT RCL] | 1.8 | 0.9 | 1.3 | 3.2 | 45.7↑ | 21.5↑ | 7.1↑ | 20.7↑ | 30.6↑ |
| Inflammation | | | | | | | | | | |
| Fibrinolysis | Fibronectin (FN1) | 223.0 | 289.0 | 298.0 | 368.0 | 373.0 | 322.0 | 68.0 | 372.0 | 97.0 |
| Pregnancy | Pregnancy Zone Protein (PZP) | 11.0 | 3.0 | 11.0 | 6.0 | | 2.0 | | 10.0 | |
| Associated | | | | | | | | | | |

TABLE 3
Quantitative levels of SERPIN proteins obtained from healthy and cancerous samples.

| | | | | | | | | Cancer | | | | |
|---|---|---|---|---|---|---|---|---|---|---|---|---|
| Protein ID | | | | | | | | | | Non- | | |
| Uniprot | Protein | | Healthy | | | | | Stage 1 | Stage | Hodgkin's | Stage 2b | |
| Gene | Description, | Reported | Subject | Subject | Subject | Subject | Subject | Breast | 2 Lung | Lym- | Pancreatic | Ovarian |
| Identifier | Common Names | Function | 1 | 2 | 3 | 4 | 5 | Cancer | Cancer | phoma | Cancer | Cancer |
| SERPINA3 | Antichymotrypsin | Apoptosis, | 153 | 190 | 182 | 191 | 227 | 227 | 239 | 271 | 279↑ | 362↑ |
| | | Inflammation | | | | | | | | | | |
| SERPING1 | Plasma Protease | Coagulation, | 142 | 198 | 231 | 249 | 243 | 243 | 216 | 259 | 248 | 263 |
| | C1 Inhibitor | Complement | | | | | | | | | | |
| SERPIND1 | Heparin Cofactor | Coagulation | 116 | 117 | 142 | 190 | 181 | 181 | 148 | 121 | 76↓ | 125 |
| | II | | | | | | | | | | | |
| SERPINC1 | Antithrombin, | Coagulation, | 51 | 28 | 41 | 35 | 38 | 38 | 51 | 75↑ | 82↑ | 40 |
| | ATIII | Angiogenesis | | | | | | | | | | |
| SERPINA1 | Alpha-1- | Inflammation | 60 | 44 | 46 | 28 | 24 | 24↓ | 31 | 66↑ | 165↑ | 70↑ |
| | Antitrypsin | | | | | | | | | | | |
| SERPINA4 | Kallistatin | Kidney | 35 | 42 | 50 | 57 | 42 | 42 | 54 | 45 | 33 | 26↓ |
| | | function, | | | | | | | | | | |
| | | Inflammation | | | | | | | | | | |
| SERPINA5 | Protein C | Coagulation, | 17 | 19 | 24 | 27 | 16 | 16 | 16 | 9↓ | 8↓ | 8↓ |
| | Inhibitor | Inflammation | | | | | | | | | | |
| SERPINA10 | Z-dependent | Coagulation | 18 | 14 | 17 | 14 | 12 | 12 | 12 | 15 | 12 | 17 |
| | proteinase | | | | | | | | | | | |
| | inhibitor | | | | | | | | | | | |
| SERPINF2 | α-2-antiplasmin | Fibrinolysis | 9 | 6 | 8 | 7 | 7 | 7 | 8 | 6 | 7 | 6 |

TABLE 4
Detected sub-populations of active AAT and inactive AAT

| | | | | Bead | | Flow-Through | | Serum | |
|---|---|---|---|---|---|---|---|---|---|
| AAT | | | | Bound | | (Unbound) | | Untreated | |
| Peptide | Start | | | TMT | Spectral | TMT | Spectral | TMT | Spectral |
| Region | position | Sequence | End position | Ratio | Count | Ratio | Count | Ratio | Count |
| Adjacent | 360 | AVLTIDEK | 367 | 0.35 | 9 | 1.78 | 21 | 1.53 | 14 |
| RCL Tryptic | | | | | | | | | |
| RCL Cleaved | 368 | GTEAAGAMF | 382 | | | 1.05 | 7 | 1.16 | 23 |
| | | LEAIPM | | | | | | | |
| RCL Intact | 368 | GTEAAGAMF | 389 | 0.77 | 5 | 1.75 | 1 | 1.34 | 50 |
| | | LEAIPMSIPPE | | | | | | | |
| | | VK | | | | | | | |
| RCL Cleaved | 383 | SIPPEVK | 389 | | | 1.45 | 27 | 1.44 | 18 |

TABLE 5
Quantitative levels of subpopulations of AAT in healthy and cancerous samples.

| | | | | | Cancer | | | | |
|---|---|---|---|---|---|---|---|---|---|
| | | | | | Stage 1 | Stage 2 | Non- | Stage 2b | |
| | Healthy | | | | Breast | Lung | Hodgkin's | Pancreatic | Ovarian |
| Description | Subject 1 | Subject 2 | Subject 3 | Subject 4 | Cancer | Cancer | Lymphoma | Cancer | Cancer |
| Inactive AAT | 12.2 | 9.9 | 9.8 | 4.7 | 4.7 | 11 | 28.4 | 8.5 | 2.6 |
| Active AAT | 6.8 | 10.8 | 7.5 | 1.5 | 0.1 | 0.5 | 4 | 0.4 | 0.1 |
| Ratio of (Inactive | 1.8 | 0.9 | 1.3 | 3.2 | 45.7↑ | 21.5↑ | 7.1↑ | 20.7↑ | 30.6↑ |
| AAT):(Active | | | | | | | | | |
| AAT) | | | | | | | | | |

TABLE 6
Biomarkers and respective accession numbers

| Biomarker | Accession Number |
|---|---|
| AAT | NM_001002235 |
| ELANE | NM_001972 |
| TIMP1 | NM_003254 |
| PPBP | NM_002704 |
| PF4 | NM_002619 |
| THBS1 | NM_003246 |
| SERPIND1 | NM_000185 |
| ECM1 | NM_004425 |
| C3 | NM_000064 |
| C4BPA | NM_000715 |
| CFP | NM_002621 |
| SAA2 | NM_030754 |
| CHGA | NM_001275 |
| FN1 | NM_212476 |
| PZP | NM_002864 |
| SERPINA3 | NM_001085 |
| SERPING1 | NM_000062 |
| SERPINC1 | NM_000488 |
| SERPINA4 | NM_006215 |
| SERPINA5 | NM_000624 |
| SERPINA10 | NM_016186 |
| SERPINF2 | NM_000934 |
| ITIH1 | NM_002215 |
| ITIH2 | NM_002216 |
| ITIH3 | NM_002217 |
| ITIH4 | NM_002218 |
| APOA1 | NM_00003 |
| APOC3 | NM_00004 |
| CRP | NM_000567 |
| CLU | NM_001831 |
| PIGR | NM_002644 |
| NAP-2 | NM_002704 |
| C2 | NM_000063 |
| C4a | NM_007293 |
| C5 | NM_001735 |
| TTR | NM_00037 |
| AGT | NM_000029 |
| CPN1 | NM_001308 |
| IGL V3-9 | NG_000002 |
| IGHV1OR15-1 | NR_135694 |
| IGHV3-53 | NG_001019 |
| IGKV1D-33 | NG_000833 |
| SHBG | NM_001040 |
| SEMA3D | NM_152754 |
| CFAP61 | NM_015585 |
| PFKM | NM_000289 |
| SPRED2 | NM_001128210 |
| C18orf63 | NM_001174123 |
| IGLV3-27 | NG_000002 |
| KNG1 | NM_001102416 |
| KLKB1 | NM_000892 |
| PSA | NM_145864 |

What is claimed is:

1. A method for treating a cancer subject, the method comprising:
obtaining a dataset comprising levels of two or more biomarker proteins in a sample obtained from the cancer subject, the two or more biomarker proteins involved in two or more interconnected pathways of dysregulation or systemic regulation of the two or more interconnected pathways of dysregulation, the two or more interconnected pathways comprising a coagulation pathway, a complement pathway, and an acute-phase inflammation pathway;
determining a disease state of the cancer based on the detected levels of the biomarker proteins; and
based on the determined disease state of the cancer, administering a therapeutic compound that modulates one or more of the detected levels of the biomarker proteins towards corresponding levels of the biomarker proteins that are exhibited by healthy subjects,
wherein biomarker proteins involved in the coagulation pathway comprise tissue inhibitor of metalloproteinases-1 (TIMP1), Pro-platelet basic protein (PPBP), thrombospondin 1 (THBS1), platelet Factor 4 (PF4), and an active subpopulation of heparin cofactor 2 (HEP2),
wherein biomarker proteins involved in the complement pathway comprise complement (C3), complement component 4 binding protein alpha (C4BPA), properdin (PROP),
wherein biomarker proteins involved in the acute-phase inflammation pathway comprise, Serum Amyloid 2 (SAA2), extracellular matrix protein 1 (ECM1), Neutrophil Elastase (ELANE), and chromogranin A (CMGA),
wherein biomarker proteins involved in the systemic regulation of the coagulation, complement, and acute-phase inflammation pathways comprise one or more serine proteinase inhibitor (SERPIN) proteins.

2. The method of claim 1, wherein the one or more SERPIN proteins comprise alpha-1-antitrypsin (SERPINA1), wherein determining the disease state of the cancer based on the detected levels of the biomarker proteins comprises:
determining a ratio of a detected level of an inactive subpopulation of SERPINA1 and a detected level of an active subpopulation of SERPINA1; and
determining that the determined ratio is elevated in comparison to a corresponding ratio of a level of an inactive subpopulation of SERPINA1 and a level of an active subpopulation of SERPINA1 detected in samples obtained from healthy subjects,
wherein the determined ratio is at least 3.5 times greater than the corresponding ratio detected in samples obtained from healthy subjects.

3. The method of claim 1, wherein the one or more SERPIN proteins are antichymotrypsin (SERPINA3), plasma protease C1 inhibitor (SERPING1), heparin cofactor II (SERPIND1), antithrombin III (SERPINC1), alpha-1-antitrypsin (SERPINA1), kallistatin (SERPINA4), protein C inhibitor (SERPINA5), Z-dependent proteinase inhibitor (SERPINA10), and alpha-2-antiplasmin (SERPINF2).

4. The method of claim 1, wherein determining the disease state of the cancer based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects.

5. The method of claim 4, wherein determining the disease state of the cancer based on the detected levels of the biomarker proteins comprises determining that a detected level of an inactive subpopulation of a SERPIN protein is at least 1.5 times greater or 1.5 times less than a level of an inactive subpopulation of a SERPIN protein detected in samples obtained from healthy subjects.

6. The method of claim 1, wherein determining the disease state of the cancer based on the detected levels of the biomarker proteins comprises determining a ratio of a detected level of ELANE to one of a detected level of an inactive subpopulation of SERPINA1 or a detected level of an active subpopulation of SERPINA1.

7. The method of claim 6, wherein the determined ratio of the detected level of ELANE to the detected level of the active subpopulation of SERPINA1 is at least 10 times greater than the corresponding ratio detected in samples obtained from healthy subjects.

8. The method of claim 1, wherein determining the disease state of the cancer based on the detected levels of the biomarker proteins further comprises determining that the detected levels of one or more of THBS1, TIMP1, PPBP, or PF4 are elevated, and the active subpopulation of HEP2 is lower in comparison to corresponding levels detected in samples obtained from healthy subjects.

9. The method of claim 8, wherein determining the disease state of the cancer based on the detected levels of the biomarker proteins further comprises determining that the detected levels of one or more of THBS1, TIMP1, PPBP, or PF4 are at least 10 times greater, and the active subpopulation HEP2 is at least 1.5 times lower in comparison to corresponding levels detected in samples obtained from healthy subjects.

10. The method of claim 1, wherein determining the disease state of the cancer based on the detected levels of the biomarker proteins further comprises determining that the detected levels of one or more of C3, C4BPA, or PROP are lower in comparison to corresponding levels detected in samples obtained from healthy subjects.

11. The method of claim 10, wherein determining the disease state of the cancer based on the detected levels of the biomarker proteins further comprises determining that the detected levels of one or more of C3, C4BPA, or PROP are at least 1.5 times lower in comparison to corresponding levels detected in samples obtained from healthy subjects.

12. The method of claim 1, wherein determining the disease state of the cancer based on the detected levels of the biomarker proteins comprises determining that a detected level of one or more of SAA2, ECM1, ELANE, and CMGA are elevated in comparison to corresponding levels detected in samples obtained from healthy subjects.

13. The method of claim 12, wherein determining the disease state of the cancer based on the detected levels of the biomarker proteins comprises determining that a detected level of one or more of SAA2 and ECM1 are elevated at least 1.5 times, ELANE is elevated at least 2 times, and CMGA is elevated at least 10 times in comparison to corresponding levels detected in samples obtained from healthy subjects.

14. A method for determining or diagnosing presence of cancer or risk factors for cancer in a subject, the method comprising:
obtaining a dataset comprising levels of two or more biomarker proteins in a sample obtained from the cancer subject, the two or more biomarker proteins involved in two or more interconnected pathways of dysregulation or systemic regulation of the two or more interconnected pathways of dysregulation, the two or more interconnected pathways comprising a coagulation pathway, a complement pathway, and an acute-phase inflammation pathway;
determining or diagnosing presence of cancer or risk factors for cancer in the subject based on the detected levels of the biomarker proteins,
wherein biomarker proteins involved in the coagulation pathway comprise tissue inhibitor of metalloproteinases-1 (TIMP1), Pro-platelet basic protein (PPBP), thrombospondin 1 (THBS1), platelet Factor 4 (PF4), and an active subpopulation of heparin cofactor 2 (HEP2),
wherein biomarker proteins involved in the complement pathway comprise complement (C3), complement component 4 binding protein alpha (C4BPA), properdin (PROP),
wherein biomarker proteins involved in the acute-phase inflammation pathway comprise, Serum Amyloid 2 (SAA2), extracellular matrix protein 1 (ECM1), Neutrophil Elastase (ELANE), and chromogranin A (CMGA),
wherein biomarker proteins involved in the systemic regulation of the coagulation, complement, and acute-phase inflammation pathways comprise one or more serine proteinase inhibitor (SERPIN) proteins.

15. The method of claim 14, wherein the one or more SERPIN proteins comprise alpha-1-antitrypsin (SERPINA1), wherein determining or diagnosing presence of cancer or risk factors for cancer in the subject based on the detected levels of the biomarker proteins comprises:
determining a ratio of a detected level of an inactive subpopulation of SERPINA1 and a detected level of an active subpopulation of SERPINA1; and
determining that the determined ratio is elevated in comparison to a corresponding ratio of a level of an inactive subpopulation of SERPINA1 and a level of an active subpopulation of SERPINA1 detected in samples obtained from healthy subjects,
wherein the determined ratio is at least 3.5 times greater than the corresponding ratio detected in samples obtained from healthy subjects.

16. The method of claim 14, wherein the one or more SERPIN proteins are antichymotrypsin (SERPINA3), plasma protease C1 inhibitor (SERPING1), heparin cofactor II (SERPIND1), antithrombin III (SERPINC1), alpha-1-antitrypsin (SERPINA1), kallistatin (SERPINA4), protein C inhibitor (SERPINA5), Z-dependent proteinase inhibitor (SERPINA10), and alpha-2-antiplasmin (SERPINF2).

17. The method of claim 14, wherein determining or diagnosing presence of cancer or risk factors for cancer in the subject based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects.

18. The method of claim 14, wherein determining or diagnosing presence of cancer or risk factors for cancer in the subject based on the detected levels of the biomarker proteins comprises determining a ratio of a detected level of an inactive subpopulation of one of the one or more SERPIN proteins to a level of an inactive subpopulation of the one SERPIN protein detected in samples obtained from healthy subjects.

19. The method of claim 14, wherein determining or diagnosing presence of cancer or risk factors for cancer in the subject based on the detected levels of the biomarker proteins comprises determining a ratio of a detected level of ELANE to one of a detected level of an inactive subpopulation of SERPINA1 or a detected level of an active subpopulation of SERPINA1.

20. The method of claim 19, wherein the determined ratio of the detected level of ELANE to the detected level of the active subpopulation of SERPINA1 is at least 10 times greater than the corresponding ratio detected in samples obtained from healthy subjects.

21. The method of claim 14, wherein determining or diagnosing presence of cancer or risk factors for cancer in the subject based on the detected levels of the biomarker proteins comprises determining that the detected levels of one or more of THBS1, TIMP1, PPBP, or PF4 are elevated, and the active subpopulation of HEP2 is lower in comparison to corresponding levels detected in samples obtained from healthy subjects.

22. The method of claim 21, wherein determining or diagnosing presence of cancer or risk factors for cancer in the subject based on the detected levels of the biomarker proteins comprises determining that the detected levels of one or more of THBS1, TIMP1, PPBP, or PF4 are at least 10 times greater, and the active subpopulation of HEP2 is at least 1.5 times lower in comparison to corresponding levels detected in samples obtained from healthy subjects.

23. The method of claim 14, wherein determining or diagnosing presence of cancer or risk factors for cancer in the subject based on the detected levels of the biomarker proteins comprises determining that the detected levels of one or more of C3, C4BPA, or PROP are lower in comparison to corresponding levels detected in samples obtained from healthy subjects.

24. The method of claim 23, wherein determining or diagnosing presence of cancer or risk factors for cancer in the subject based on the detected levels of the biomarker proteins comprises determining that the detected levels of one or more of C3, C4BPA, or PROP are at least 1.5 times lower in comparison to corresponding levels detected in samples obtained from healthy subjects.

25. The method of claim 14, wherein determining or diagnosing presence of cancer or risk factors for cancer in the subject based on the detected levels of the biomarker proteins comprises determining that a detected level of one or more of SAA2, ECM1, ELANE and CMGA are elevated in comparison to corresponding levels detected in samples obtained from healthy subjects.

26. The method of claim 25, wherein determining or diagnosing presence of cancer or risk factors for cancer in the subject based on the detected levels of the biomarker proteins comprises determining that a detected level of one or more of SAA2 and ECM1 are elevated at least 1.5 times, ELANE is elevated at least 2 times, and CMGA is elevated at least 10 times in comparison to corresponding levels detected in samples obtained from healthy subjects.
